(19) United States
(12) Patent Application Publication
(10) Pub. No.: US 2024/0138919 A1
(43) Pub. Date: May 2, 2024

(54) SYSTEMS AND METHODS FOR SELECTING, REVIEWING, MODIFYING, AND/OR APPROVING SURGICAL PLANS

(71) Applicant: Carlsmed, Inc., Carlsbad, CA (US)

(72) Inventors: Niall Patrick Casey, Carlsbad, CA (US); Jeffery Pennal, San Diego, CA (US); Matthew Chen, San Diego, CA (US)

(21) Appl. No.: 18/384,762

(22) Filed: Oct. 27, 2023

Related U.S. Application Data
(60) Provisional application No. 63/420,279, filed on Oct. 28, 2022.

(51) Int. Cl. A61B 34/10; A61F 2/46; G16H 20/40; G16H 50/50

(52) U.S. Cl. CPC A61B 34/10; A61F 2/4611; G16H 20/40; G16H 50/50; A61B 2034/104; A61B 2034/105; A61B 2034/108; A61F 2002/4633

(57) ABSTRACT

Systems and methods for reviewing and analyzing proposed patient-specific surgical plans are described herein. In some embodiments, a surgical plan review program provides a user-friendly interface for a surgeon to review various aspects of a proposed surgical plan. The program enables the surgeon to approve the surgical plan and/or provide feedback on the surgical plan to prompt revisions to the surgical plan.

### CROSS-REFERENCE TO RELATED APPLICATION

The present application claims priority to U.S. Provisional Patent Application No. 63/420,279, filed Oct. 28, 2022, the disclosure of which is incorporated by reference herein in its entirety.

### TECHNICAL FIELD

The present disclosure is generally related to designing and implementing medical care, and more particularly to systems and methods for designing and implementing patient-specific surgical procedures and/or medical devices.

### BACKGROUND

Surgical procedures to implant orthopedic implants are used to correct numerous different maladies in a variety of contexts, including spine surgery, hand surgery, shoulder and elbow surgery, total joint reconstruction (arthroplasty), skull reconstruction, pediatric orthopedics, foot and ankle surgery, musculoskeletal oncology, surgical sports medicine, and orthopedic trauma. Spine surgery itself may encompass a variety of procedures and targets, such as one or more of the cervical spine, thoracic spine, lumbar spine, or sacrum, and may be performed to treat a deformity or degeneration of the spine and/or related back pain, leg pain, or other body pain. Common spinal deformities that may be treated using an orthopedic implant include irregular spinal curvature such as scoliosis, lordosis, or kyphosis (hyper- or hypo-), and irregular spinal displacement (e.g., spondylolisthesis). Other spinal disorders that can be treated using an orthopedic implant include osteoarthritis, lumbar degenerative disc disease or cervical degenerative disc disease, lumbar spinal stenosis, and cervical spinal stenosis.

### BRIEF DESCRIPTION OF THE DRAWINGS

The accompanying drawings illustrate various embodiments of systems, methods, and embodiments of various other aspects of the disclosure. Any person with ordinary skill in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 is a network connection diagram illustrating a system for providing patient-specific medical care in accordance with embodiments of the present technology.

FIG. 2 illustrates a computing device suitable for use in connection with the system of FIG. 1 in accordance with embodiments of the present technology.

FIG. 3 is a flow diagram illustrating a method for providing patient-specific medical care in accordance with embodiments of the present technology.

FIG. 4 is a flow diagram illustrating another method for providing patient-specific medical care in accordance with embodiments of the present technology.

FIGS. 5A-5E illustrate various aspects of a case display dashboard generated using a surgical plan review program in accordance with embodiments of the present technology.

FIGS. 6A-12 illustrate various aspects of a surgical plan review dashboard generated using a surgical plan review program in accordance with embodiments of the present technology.

### DETAILED DESCRIPTION

The present technology is directed to systems and methods for planning and implementing medical procedures and/or devices. For example, in many of the embodiments disclosed herein, a method of providing medical care includes generating a patient-specific surgical plan that can provide a recommended treatment protocol for a particular patient. The surgical plan may include a surgical intervention type, a surgery location, and predicted post-operative data associated with the corresponding surgical intervention and/or surgical location, among other information. The systems and methods described herein further enable a surgeon or other healthcare provider to review and optionally provide feedback on the proposed patient-specific surgical plan. For example, the present technology provides a surgical plan review program that can be implemented on a smart phone, tablet, or other computing device and used by a surgeon or other user to track, review, and analyze a proposed surgical plan. The surgical plan review program can also enable a surgeon to provide feedback on a proposed surgical plan and/or approve a proposed surgical plan. Without intending to be bound by theory, the surgical plan review program is expected to provide a single, user-friendly interface that enables a surgeon or other user to easily track multiple separate patient cases, and easily review, comment on, and approve proposed surgical plans. In this way, the surgical plan review programs described throughout this Detailed Description are expected to improve case management, thoroughness of surgeon review of proposed surgical plans, efficiency of surgeon review of proposed surgical plans, patient outcomes, and the like.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

As used herein and in the appended claims, the singular forms “a,” “an,” and “the” include plural references unless the context clearly dictates otherwise.

Although the disclosure herein primarily describes systems and methods for treatment planning in the context of orthopedic surgery, the technology may be applied equally to medical treatment and devices in other fields (e.g., other types of surgical practice). Additionally, although many embodiments herein describe systems and methods with respect to implanted devices, the technology may be applied equally to other types of medical devices (e.g., non-implanted devices).

The headings are provided for convenience only and should not be used to interpret the scope of the present technology.

### A. Select Embodiments of Systems for Designing Patient-Specific Surgical Plans and Patient-Specific Implants

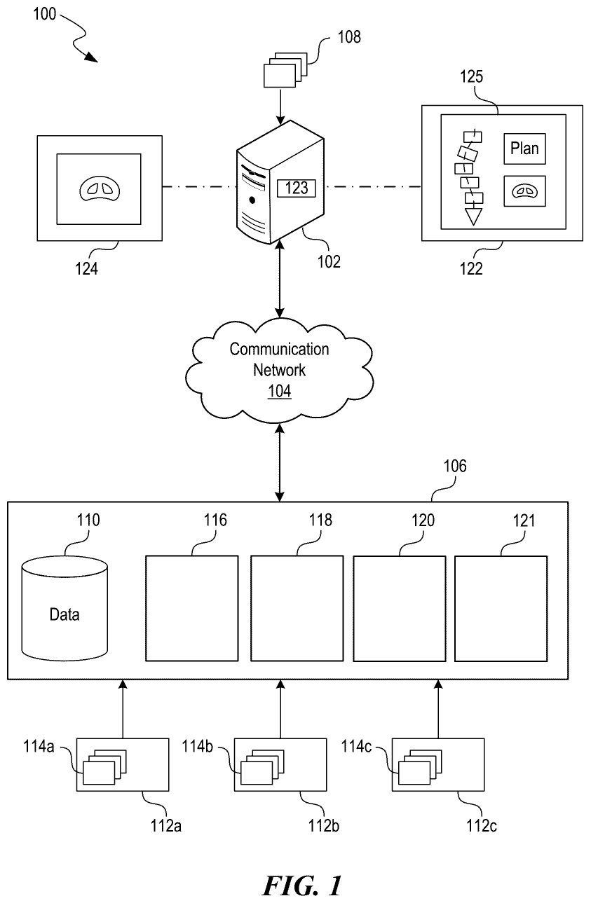

FIG. 1 is a network connection diagram illustrating a system 100 for providing patient-specific medical care, according to embodiments of the present technology. As described in further detail herein, the system 100 is configured to generate a medical treatment plan for a patient. In some embodiments, the system 100 is configured to generate a medical treatment plan for a patient suffering from an orthopedic or spinal disease or disorder, such as trauma (e.g., fractures), cancer, deformity, degeneration, pain (e.g., back pain, leg pain), irregular spinal curvature (e.g., scoliosis, lordosis, kyphosis), irregular spinal displacement (e.g., spondylolisthesis, lateral displacement axial displacement), osteoarthritis, lumbar degenerative disc disease, cervical degenerative disc disease, lumbar spinal stenosis, or cervical spinal stenosis, or a combination thereof. The medical treatment plan can include surgical information, technology recommendations (e.g., device and/or instrument recommendations), and/or medical device designs. For example, the medical treatment plan can include at least surgical procedure (e.g., a surgical procedure or intervention) and/or at least one medical device (e.g., an implanted medical device (also referred to herein as an “implant” or “implanted device”) or implant delivery instrument). In some embodiments, the medical treatment plan is therefore also referred to as a “surgical plan,” a “patient-specific surgical plan,” a “patient-specific treatment plan,” or the like.

In some embodiments, the system 100 generates a medical treatment plan that is customized for a particular patient or group of patients, also referred to herein as a “patient-specific” or “personalized” treatment or surgical plan. The patient-specific surgical plan can include at least one patient-specific surgical procedure and/or at least one patient-specific medical device that are designed and/or optimized for the patient's particular characteristics (e.g., condition, anatomy, pathology, condition, medical history). For example, the patient-specific medical device can be designed and manufactured specifically for the particular patient, rather than being an off-the-shelf device. However, it shall be appreciated that a patient-specific surgical plan can also include aspects that are not customized for the particular patient. For example, a patient-specific or personalized surgical procedure can include one or more instructions, portions, steps, etc. that are non-patient-specific. Likewise, a patient-specific or personalized medical device can include one or more components that are non-patient-specific, and/or can be used with an instrument or tool that is non-patient-specific. Personalized implant designs can be used to manufacture or select patient-specific technologies, including medical devices, instruments, and/or surgical kits. For example, a personalized surgical kit can include one or more patient-specific devices, patient-specific instruments, non-patient-specific technology (e.g., standard instruments, devices, etc.), instructions for use, patient-specific treatment plan information, or a combination thereof.

The system 100 includes a client computing device 102, which can be a user device, such as a smart phone, mobile device, laptop, desktop, personal computer, tablet, phablet, or other such devices known in the art. As discussed further herein, the client computing device 102 can include one or more processors, and memory storing instructions executable by the one or more processors to perform the methods described herein. The client computing device 102 can be associated with a healthcare provider (e.g., a surgeon, healthcare administrator, hospital system, etc.) that is treating the patient. Although FIG. 1 illustrates a single client computing device 102, in alternative embodiments, the client computing device 102 can instead be implemented as a client computing system encompassing a plurality of computing devices, such that the operations described herein with respect to the client computing device 102 can instead be performed by the client computing system and/or the plurality of client computing devices.

The client computing device 102 is configured to receive a patient data set 108 associated with a patient to be treated. The patient data set 108 can include data representative of the patient's condition, anatomy, pathology, medical history, preferences, and/or any other information or parameters relevant to the patient. For example, the patient data set 108 can include medical history, surgical intervention data, treatment outcome data, progress data (e.g., physician notes), patient feedback (e.g., feedback acquired using quality of life questionnaires, surveys), clinical data, provider information (e.g., physician, hospital, surgical team), patient information (e.g., demographics, sex, age, height, weight, type of pathology, occupation, activity level, tissue information, health rating, comorbidities, health related quality of life (HRQL)), vital signs, diagnostic results, medication information, allergies, image data (e.g., camera images, Magnetic Resonance Imaging (MRI) images, ultrasound images, Computerized Aided Tomography (CAT) scan images, Positron Emission Tomography (PET) images, X-Ray images), diagnostic equipment information (e.g., manufacturer, model number, specifications, user-selected settings/configurations, etc.), or the like. In some embodiments, the patient data set 108 includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine.

The client computing device 102 is also configured to enable a user (e.g., a surgeon) to review one or more proposed surgical plans for a patient to be treated. In particular, the client computing device 102 can include a surgical plan review software module 123 (“the review module 123”). As described in detail below and throughout this Detailed Description, the review module 123 can comprise computer-executable instructions for generating, displaying, and/or implementing a surgical plan review program or platform 125 (“the review program 125”) that facilitates surgeon or user review of one or more patient-specific surgical plans via the client computing device 102.

The review module 123 can be stored in the form of computer-readable or computer-executable instructions on a memory (not shown) of the client computing device 102. In other embodiments, the review module 123 can be stored remotely from the client computing device 102 (e.g., in the cloud or at a remote server) and implemented on the client computing device 102 via a remote (e.g., wireless) connection. In yet other embodiments, some of the review module 123 can be stored locally at the client computing device 102 while other aspects of the review module 123 can be store remotely.

The client computing device 102 is operably connected via a communication network 104 to a server 106, thus allowing for data transfer between the client computing device 102 and the server 106. The communication network 104 may be a wired and/or a wireless network. The communication network 104, if wireless, may be implemented using communication techniques such as Visible Light Communication (VLC), Worldwide Interoperability for Microwave Access (WiMAX), Long term evolution (LTE), Wireless local area network (WLAN), Infrared (IR) communication, Public Switched Telephone Network (PSTN), Radio waves, and/or other communication techniques known in the art.

The server 106, which may also be referred to as a “treatment assistance network” or “prescriptive analytics network,” can include one or more computing devices and/or systems. As discussed further herein, the server 106 can include one or more processors, and memory storing instructions executable by the one or more processors to perform some or all of the methods described herein. In some embodiments, the server 106 is implemented as a distributed “cloud” computing system or facility across any suitable combination of hardware and/or virtual computing resources.

The client computing device 102 and server 106 can individually or collectively perform some or all of the various methods described herein for providing patient-specific medical care. For example, some or all of the steps of the methods described herein can be performed by the client computing device 102 alone, the server 106 alone, or a combination of the client computing device 102 and the server 106. Thus, although certain operations are described herein with respect to the server 106, it shall be appreciated that these operations can also be performed by the client computing device 102, and vice-versa, unless the context clearly dictates otherwise.

The server 106 includes at least one database 110 configured to store reference data useful for the treatment planning methods described herein. The reference data can include historical and/or clinical data from the same or other patients, data collected from prior surgeries and/or other treatments of patients by the same or other healthcare providers, data relating to medical device designs, data collected from study groups or research groups, data from practice databases, data from academic institutions, data from implant manufacturers or other medical device manufacturers, data from imaging studies, data from simulations, clinical trials, demographic data, treatment data, outcome data, mortality rates, or the like.

In some embodiments, the database 110 includes a plurality of reference patient data sets, each patient reference data set associated with a corresponding reference patient. For example, the reference patient can be a patient that previously received treatment or is currently receiving treatment. Each reference patient data set can include data representative of the corresponding reference patient's condition, anatomy, pathology, medical history, disease progression, preferences, and/or any other information or parameters relevant to the reference patient, such as any of the data described herein with respect to the patient data set 108. In some embodiments, the reference patient data set includes pre-operative data, intra-operative data, and/or post-operative data. For example, a reference patient data set can include data representing one or more of patient ID, age, gender, BMI, lumbar lordosis, Cobb angle(s), pelvic incidence, disc height, segment flexibility, bone quality, rotational displacement, and/or treatment level of the spine. As another example, a reference patient data set can include treatment data regarding at least one surgical procedure performed on the reference patient, such as descriptions of surgical procedures or interventions (e.g., surgical approaches, bony resections, surgical maneuvers, corrective maneuvers, placement of implants or other devices). In some embodiments, the treatment data includes medical device design data for at least one medical device used to treat the reference patient, such as physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties). In yet another example, a reference patient data set can include outcome data representing an outcome of the treatment of the reference patient, such as corrected anatomical metrics, presence of fusion, HRQL, pain level, activity level, return to work, complications, recovery times, efficacy, mortality, and/or follow-up surgeries.

In some embodiments, the server 106 receives at least some of the reference patient data sets from a plurality of healthcare provider computing systems (e.g., systems 112a-112c, collectively 112). The server 106 can be connected to the healthcare provider computing systems 112 via one or more communication networks (not shown). Each healthcare provider computing system 112 can be associated with a corresponding healthcare provider (e.g., physician, surgeon, medical clinic, hospital, healthcare network, etc.). Each healthcare provider computing system 112 can include at least one reference patient data set (e.g., reference patient data sets 114a-114c, collectively 114) associated with reference patients treated by the corresponding healthcare provider. The reference patient data sets 114 can include, for example, electronic medical records, electronic health records, biomedical data sets, etc. The reference patient data sets 114 can be received by the server 106 from the healthcare provider computing systems 112 and can be reformatted into different formats for storage in the database 110. Optionally, the reference patient data sets 114 can be processed (e.g., cleaned) to ensure that the represented patient parameters are likely to be useful in the treatment planning methods described herein.

As described in further detail herein, the server 106 can be configured with one or more algorithms that generate patient-specific surgical plan data (e.g., treatment procedures, target anatomical corrections, medical devices, etc.) based on the reference data. In some embodiments, the patient-specific data is generated based on correlations between the patient data set 108 and the reference data. Optionally, the server 106 can predict outcomes, including recovery times, efficacy based on clinical end points, likelihood of success, predicted mortality, predicted related follow-up surgeries, or the like. In some embodiments, the server 106 can continuously or periodically analyze patient data (including patient data obtained during the patient stay) to determine near real-time or real-time risk scores, mortality prediction, etc.

In some embodiments, the server 106 includes one or more modules for performing one or more steps of the patient-specific treatment planning methods described herein. For example, in the depicted embodiment, the server 106 includes a data analysis module 116, a treatment planning module 118, a disease progression module 120, and an intervention timing module 121. In alternative embodiments, one or more of these modules may be combined with each other, or may be omitted. Thus, although certain operations are described herein with respect to a particular module or modules, this is not intended to be limiting, and such operations can be performed by a different module or modules in alternative embodiments.

The data analysis module 116 is configured with one or more algorithms for identifying a subset of reference data from the database 110 that is likely to be useful in developing a patient-specific treatment plan. For example, the data analysis module 116 can compare patient-specific data (e.g., the patient data set 108 received from the client computing device 102) to the reference data from the database 110 (e.g., the reference patient data sets) to identify similar data (e.g., one or more similar patient data sets in the reference patient data sets). The comparison can be based on one or more parameters, such as age, gender, BMI, lumbar lordosis, pelvic incidence, and/or treatment levels. The parameter(s) can be used to calculate a similarity score for each reference patient. The similarity score can represent a statistical correlation between the patient data set 108 and the reference patient data set. Accordingly, similar patients can be identified based on whether the similarity score is above, below, or at a specified threshold value. For example, as described in greater detail below, the comparison can be performed by assigning values to each parameter and determining the aggregate difference between the subject patient and each reference patient. Reference patients whose aggregate difference is below a threshold can be considered to be similar patients.

The data analysis module 116 can further be configured with one or more algorithms to select a subset of the reference patient data sets, e.g., based on similarity to the patient data set 108 and/or treatment outcome of the corresponding reference patient. For example, the data analysis module 116 can identify one or more similar patient data sets in the reference patient data sets, and then select a subset of the similar patient data sets based on whether the similar patient data set includes data indicative of a favorable or desired treatment outcome. The outcome data can include data representing one or more outcome parameters, such as corrected anatomical metrics, presence of fusion, HRQL, activity level, complications, recovery times, efficacy, mortality, or follow-up surgeries. As described in further detail below, in some embodiments, the data analysis module 116 calculates an outcome score by assigning values to each outcome parameter. A patient can be considered to have a favorable outcome if the outcome score is above, below, or at a specified threshold value.

In some embodiments, the data analysis module 116 selects a subset of the reference patient data sets based at least in part on user input (e.g., from a clinician, surgeon, physician, healthcare provider). For example, the user input can be used in identifying similar patient data sets. In some embodiments, weighting of similarity and/or outcome parameters can be selected by a healthcare provider or physician to adjust the similarity and/or outcome score based on clinician input. In further embodiments, the healthcare provider or physician can select the set of similarity and/or outcome parameters (or define new similarity and/or outcome parameters) used to generate the similarity and/or outcome score, respectively.

In some embodiments, the data analysis module 116 includes one or more algorithms used to select a set or subset of the reference patient data sets based on criteria other than patient parameters. For example, the one or more algorithms can be used to select the subset based on healthcare provider parameters (e.g., based on healthcare provider ranking/scores such as hospital/physician expertise, number of procedures performed, hospital ranking, etc.) and/or healthcare resource parameters (e.g., diagnostic equipment, facilities, surgical equipment such as surgical robots), or other non-patient related information that can be used to predict outcomes and risk profiles for procedures for the present healthcare provider. For example, reference patient data sets with images captured from similar diagnostic equipment can be aggregated to reduce or limit irregularities due to variation between diagnostic equipment. Additionally, patient-specific treatment plans can be developed for a particular health-care provider using data from similar healthcare providers (e.g., healthcare providers with traditionally similar outcomes, physician expertise, surgical teams, etc.). In some embodiments, reference healthcare provider data sets, hospital data sets, physician data sets, surgical team data sets, post-treatment data set, and other data sets can be utilized. By way of example, a patient-specific surgical plan to perform a battlefield surgery can be based on reference patient data from similar battlefield surgeries and/or datasets associated with battlefield surgeries. In another example, the patient-specific surgical plan can be generated based on available robotic surgical systems. The reference patient data sets can be selected based on patients that have been operated on using comparable robotic surgical systems under similar conditions (e.g., size and capabilities of surgical teams, hospital resources, etc.).

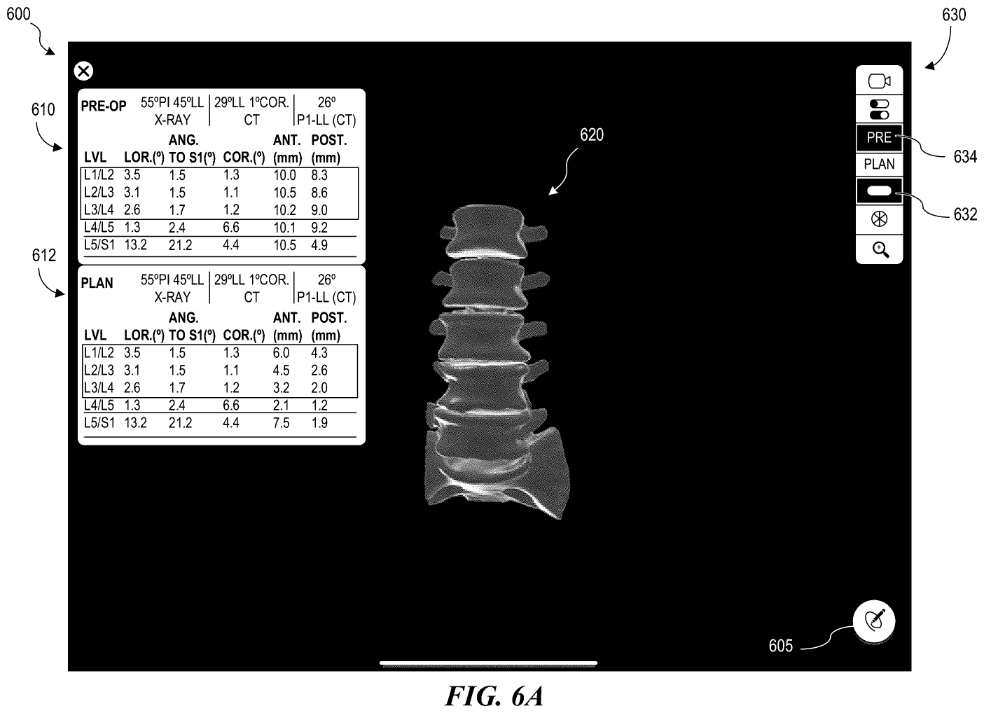

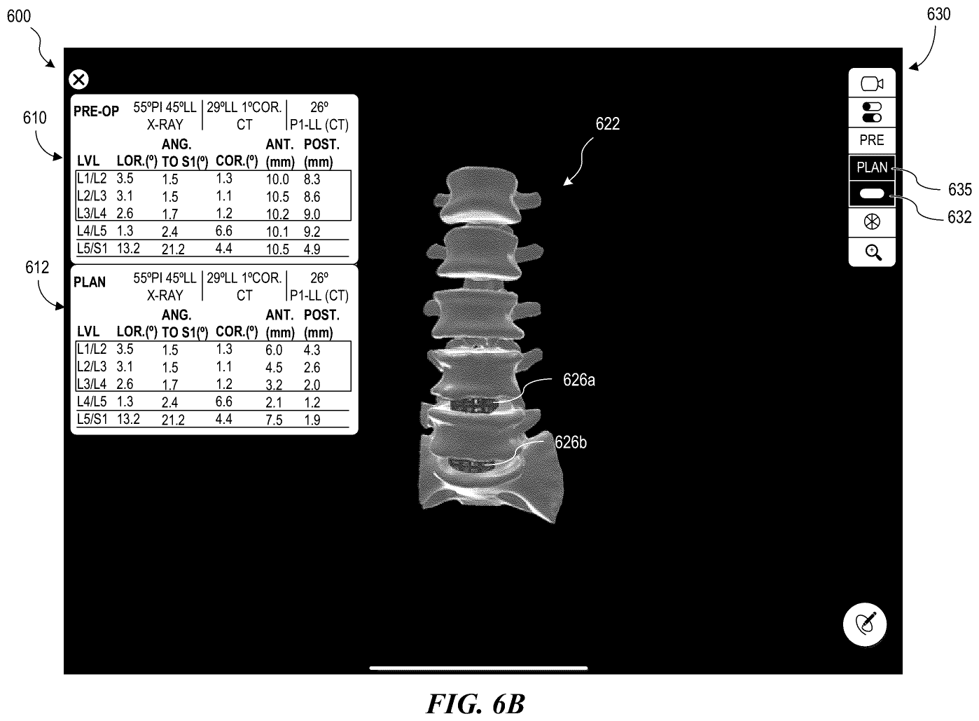

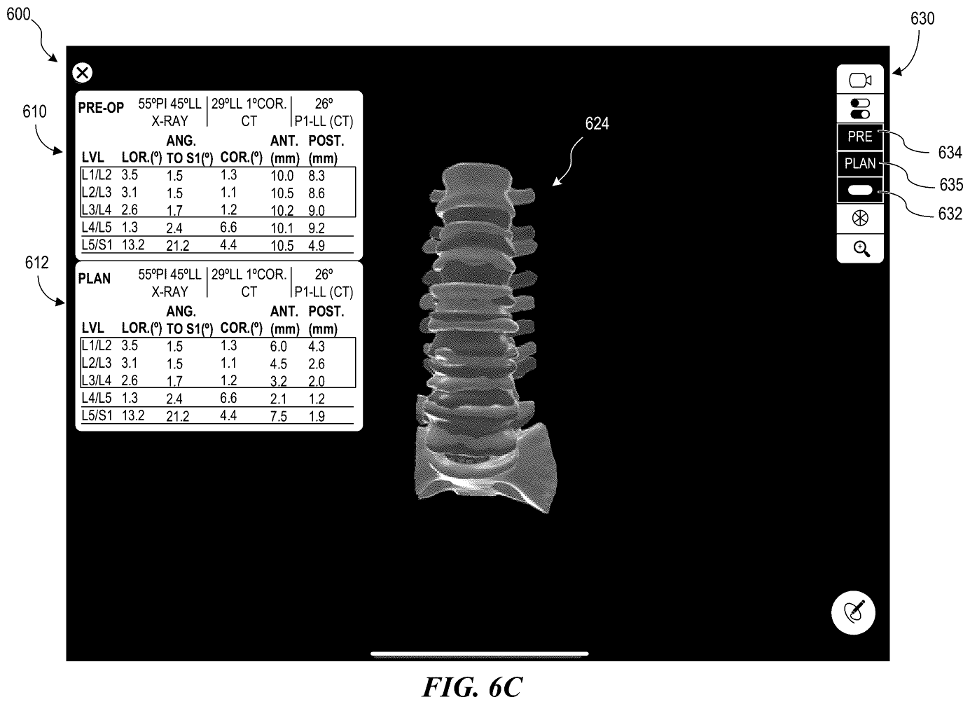

In some embodiments, the analysis module 116 is or includes a comparator configured to generate one or more visual comparisons of anatomical models, such as a comparison of a pre-operative anatomical model and a predicted post-operative anatomical model. As described in greater detail below, a virtual model of pre-operative and/or predicted post-operative patient anatomy can be generated by analyzing images of a patient. The image analysis can include, for example, segmentation processes, boundary detection processes, tissue analysis, predicted tissue manipulation, predicted tissue response, or combinations thereof. The system can generate relationships between imaged anatomical elements to generate a positioning map of the patient's anatomy. To compare multiple anatomical models, the analysis module 116 can identify keying features of corresponding anatomical models of the patient. The analysis module 116 can then overlay, overlap, or mate the models based on alignment of those keying features. This allows a user to view differences between models (e.g., differences between a pre-operative anatomical model and a predicted post-operative anatomical model), as discussed in connection with FIG. 6C. In some embodiments, the analysis module 116 can digitally overlay multiple three dimensional virtual models to generate visual comparisons. The analysis module 116 can identify and/or label differences between the models (e.g., differences meeting an threshold score).

The treatment planning module 118 is configured with one or more algorithms to generate at least one surgical plan (e.g., pre-operative plans, intra-operative plans, post-operative plans etc.) based on the output from the data analysis module 116. In some embodiments, the treatment planning module 118 is configured to develop and/or implement at least one predictive model for generating the patient-specific treatment plan, also known as a “prescriptive model.” The predictive model(s) can be developed using clinical knowledge, statistics, machine learning, AI, neural networks, or the like. In some embodiments, the output from the data analysis module 116 is analyzed (e.g., using statistics, machine learning, neural networks, AI) to identify correlations between data sets, patient parameters, healthcare provider parameters, healthcare resource parameters, treatment procedures, medical device designs, and/or treatment outcomes. These correlations can be used to develop at least one predictive model that predicts the likelihood that a surgical plan will produce a favorable outcome for the particular patient. The predictive model(s) can be validated, e.g., by inputting data into the model(s) and comparing the output of the model to the expected output.

In some embodiments, the treatment planning module 118 is configured to generate the surgical plan based on previous treatment data from reference patients. For example, the treatment planning module 118 can receive a selected subset of reference patient data sets and/or similar patient data sets from the data analysis module 116, and determine or identify treatment data from the selected subset. The treatment data can include, for example, treatment procedure data (e.g., surgical procedure or intervention data) and/or medical device design data (e.g., implant design data) that are associated with favorable or desired treatment outcomes for the corresponding patient. The treatment planning module 118 can analyze the treatment procedure data and/or medical device design data to determine an optimal treatment protocol for the patient to be treated. For example, the treatment procedures and/or medical device designs can be assigned values and aggregated to produce a treatment score. The patient-specific surgical plan can be determined by selecting surgical plan(s) based on the score (e.g., higher or highest score; lower or lowest score; score that is above, below, or at a specified threshold value). The personalized patient-specific surgical plan can be based on, at least in part, the patient-specific technologies or patient-specific selected technology.

Alternatively or in combination, the treatment planning module 118 can generate the surgical plan based on correlations between data sets. For example, the treatment planning module 118 can correlate treatment procedure data and/or medical device design data from similar patients with favorable outcomes (e.g., as identified by the data analysis module 116). Correlation analysis can include transforming correlation coefficient values to values or scores. The values/scores can be aggregated, filtered, or otherwise analyzed to determine one or more statistical significances. These correlations can be used to determine surgical procedure(s) and/or medical device design(s) that are optimal or likely to produce a favorable outcome for the patient to be treated.

Alternatively or in combination, the treatment planning module 118 can generate the surgical plan using one or more AI techniques. AI techniques can be used to develop computing systems capable of simulating aspects of human intelligence, e.g., learning, reasoning, planning, problem solving, decision making, etc. AI techniques can include, but are not limited to, case-based reasoning, rule-based systems, artificial neural networks, decision trees, support vector machines, regression analysis, Bayesian networks (e.g., naïve Bayes classifiers), genetic algorithms, cellular automata, fuzzy logic systems, multi-agent systems, swarm intelligence, data mining, machine learning (e.g., supervised learning, unsupervised learning, reinforcement learning), and hybrid systems.

In some embodiments, the treatment planning module 118 generates the surgical plan using one or more trained machine learning models. Various types of machine learning models, algorithms, and techniques are suitable for use with the present technology. In some embodiments, the machine learning model is initially trained on a training data set, which is a set of examples used to fit the parameters (e.g., weights of connections between “neurons” in artificial neural networks) of the model. For example, the training data set can include any of the reference data stored in database 110, such as a plurality of reference patient data sets or a selected subset thereof (e.g., a plurality of similar patient data sets).

In some embodiments, the machine learning model (e.g., a neural network or a naïve Bayes classifier) may be trained on the training data set using a supervised learning method (e.g., gradient descent or stochastic gradient descent). The training dataset can include pairs of generated “input vectors” with the associated corresponding “answer vector” (commonly denoted as the target). The current model is run with the training data set and produces a result, which is then compared with the target, for each input vector in the training data set. Based on the result of the comparison and the specific learning algorithm being used, the parameters of the model are adjusted. The model fitting can include both variable selection and parameter estimation. The fitted model can be used to predict the responses for the observations in a second data set called the validation data set. The validation data set can provide an unbiased evaluation of a model fit on the training data set while tuning the model parameters. Validation data sets can be used for regularization by early stopping, e.g., by stopping training when the error on the validation data set increases, as this may be a sign of overfitting to the training data set. In some embodiments, the error of the validation data set error can fluctuate during training, such that ad-hoc rules may be used to decide when overfitting has truly begun. Finally, a test data set can be used to provide an unbiased evaluation of a final model fit on the training data set.

To generate a surgical plan, the patient data set 108 can be input into the trained machine learning model(s). Additional data, such as the selected subset of reference patient data sets and/or similar patient data sets, and/or treatment data from the selected subset, can also be input into the trained machine learning model(s). The trained machine learning model(s) can then calculate whether various candidate treatment procedures and/or medical device designs are likely to produce a favorable outcome for the patient. Based on these calculations, the trained machine learning model(s) can select at least one surgical plan for the patient. In some embodiments, the trained machine learning model(s) can determine candidate procedures (or candidate surgical plans), analyze the candidate procedures, select the candidate surgical plans or portions thereof, score plans, and/or generate surgical plans for the patient. Each surgical plan can be scored (e.g., scored based on favorable outcome, likelihood of outcome, etc.) and ranked according to the score. The trained machine learning model(s) can determine a set of surgical plans that meet selection criteria for plan review by a user. The selection criteria can be based on, for example, regulatory requirements, reimbursement criteria, healthcare/provider expertise, available surgical equipment, manufacturing capabilities, elimination criteria, combinations thereof, or the like. A user can input one or more selection criteria to control the types and/or features of the surgical plans for comparison. In embodiments where multiple trained machine learning models are used, the models can be run sequentially or concurrently to compare outcomes and can be periodically updated using training data sets. The treatment planning module 118 can use one or more of the machine learning models based the model's predicted accuracy score.

The patient-specific surgical plan generated by the treatment planning module 118 can include at least one patient-specific surgical procedure (e.g., a surgical procedure or intervention) and/or at least one patient-specific medical device (e.g., an implant or implant delivery instrument). A patient-specific surgical plan can include an entire surgical procedure or portions thereof. Additionally, one or more patient-specific medical devices can be specifically selected or designed for the corresponding surgical procedure, thus allowing for the various components of the patient-specific technology to be used in combination to treat the patient.

In some embodiments, the patient-specific surgical procedure includes an orthopedic surgery procedure, such as spinal surgery, hip surgery, knee surgery, jaw surgery, hand surgery, shoulder surgery, elbow surgery, total joint reconstruction (arthroplasty), skull reconstruction, foot surgery, or ankle surgery. Spinal surgery can include spinal fusion surgery, such as posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). In some embodiments, the patient-specific treatment procedure includes descriptions of and/or instructions for performing one or more aspects of a patient-specific surgical procedure. For example, the patient-specific surgical procedure can include one or more of a surgical approach, a corrective maneuver, a bony resection, or implant placement.

In some embodiments, the patient-specific medical device design includes a design for an orthopedic implant and/or a design for an instrument for delivering an orthopedic implant. Examples of such implants include, but are not limited to, screws (e.g., bone screws, spinal screws, pedicle screws, facet screws), interbody implant devices (e.g., intervertebral implants), cages, plates, rods, disks, fusion devices, spacers, rods, expandable devices, stents, brackets, ties, scaffolds, fixation device, anchors, nuts, bolts, rivets, connectors, tethers, fasteners, joint replacements, hip implants, or the like. Examples of instruments include, but are not limited to, screw guides, cannulas, ports, catheters, insertion tools, or the like.

A patient-specific medical device design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of a corresponding medical device. For example, a design for an orthopedic implant can include implant shape, size, material, and/or effective stiffness (e.g., lattice density, number of struts, location of struts, etc.). In some embodiments, the generated patient-specific medical device design is a design for an entire device. Alternatively, the generated design can be for one or more components of a device, rather than the entire device.

In some embodiments, the design is for one or more patient-specific device components that can be used with standard, off-the-shelf components. For example, in a spinal surgery, a pedicle screw kit can include both standard components and patient-specific customized components. In some embodiments, the generated design is for a patient-specific medical device that can be used with a standard, off-the-shelf delivery instrument. For example, the implants (e.g., screws, screw holders, rods) can be designed and manufactured for the patient, while the instruments for delivering the implants can be standard instruments. This approach allows the components that are implanted to be designed and manufactured based on the patient's anatomy and/or surgeon's preferences to enhance treatment. The patient-specific devices described herein are expected to improve delivery into the patient's body, placement at the treatment site, and/or interaction with the patient's anatomy.

In embodiments where the patient-specific surgical plan includes a specific surgical procedure to implant a medical device, the treatment planning module 118 can also store various types of implant surgery information, such as implant parameters (e.g., types, dimensions), availability of implants, aspects of a pre-operative plan (e.g., initial implant configuration, detection and measurement of the patient's anatomy, etc.), FDA requirements for implants (e.g., specific implant parameters and/or characteristics for compliance with FDA regulations), or the like. In some embodiments, the treatment planning module 118 can convert the implant surgery information into formats useable for machine-learning based models and algorithms. For example, the implant surgery information can be tagged with particular identifiers for formulas or can be converted into numerical representations suitable for supplying to the trained machine learning model(s). The treatment planning module 118 can also store information regarding the patient's anatomy, such as two- or three-dimensional images or models of the anatomy, and/or information regarding the biology, geometry, and/or mechanical properties of the anatomy. The anatomy information can be used to inform implant design and/or placement.

The disease progression module 120 can be used to analyze, predict, and/or model disease progression for a particular patient. As described in detail below, the disease progression module 120 can estimate the rate of disease progression for the patient under a variety of different circumstances, including (a) if no surgical intervention occurs, and (b) if one or more surgical plans (e.g., surgical procedures identified by the treatment planning module 118) are performed. The disease progression module 120 can therefore include an algorithm, machine learning model, or other software analytical tool for predicting disease progression in a particular patient.

In some embodiments, the disease progression module 120 includes a machine learning model or other software module that can be trained based off a plurality of reference patient data sets that includes, in addition to the patient data described above, disease progression metrics for each of the reference patients. The progression metrics can include measurements for disease metrics over a period of time. Suitable metrics may include spinopelvic parameters (e.g., lumbar lordosis, pelvic tilt, sagittal vertical axis (SVA), cobb angel, coronal offset, etc.), disability scores, functional ability scores, flexibility scores, VAS pain scores, or the like. The progression of the metrics for each reference patient can be correlated to other patient information for the specific reference patient (e.g., age, sex, height, weight, activity level, diet, etc.). The disease metrics can include values over a period of time. For example, the reference patient data may include values of disease metrics on a daily, weekly, monthly, bi-monthly, yearly, or other basis. By measuring the metrics over a period of time, changes in the values of the metrics can be tracked as an estimate of disease progression and correlated to other patient data.

In some embodiments, the disease progression module 120 can therefore estimate the rate of disease progression for a particular patient. The progression may be estimated by providing estimated changes in one or more disease metrics over a period of time (e.g., X % increase in a disease metric per year). The rate can be constant (e.g., 5% increase in pelvic tilt per year) or variable (e.g., 5% increase in pelvic tilt for a first year, 10% increase in pelvic tilt for a second year, etc.). In some embodiments, the estimated rate of progression can be transmitted to a surgeon or other healthcare provider as part of a surgical plan, as described in greater detail below.

As a non-limiting example, a particular patient who is a fifty-five-year-old male may have a SVA value of 6 mm. The disease progression module 120 can analyze patient reference data sets to identify disease progression for individual reference patients having one or more similarities with the particular patient (e.g., individual patients of the reference patients who have an SVA value of about 6 mm and are approximately the same age, weight, height, and/or sex of the patient). Based on this analysis, the disease progression module 120 can predict the rate of disease progression if no surgical intervention occurs (e.g., the patient's VAS pain scores may increase 5%, 10%, or 15% annually if no surgical intervention occurs, the SVA value may continue to increase by 5% annually if no surgical intervention occurs, etc.).

The surgical treatment plans and/or associated patient-specific implants described herein can also be at least partially based on the estimated rates of disease progression, enabling the modeling of different outcomes over a desired period of times. Additionally, the models/simulations can account for any number of additional diseases or conditions to predict the patient's overall health, mobility, or the like. These additional diseases or conditions can, in combination with other patient health factors (e.g., height, weight, age, activity level, etc.) be used to generate a patient health score reflecting the overall health of the patient. The patient health score can be displayed for surgeon review and/or incorporated into the estimation of disease progression. Accordingly, the present technology can generate one or more virtual simulations of the predicted disease progression to demonstrate how the patient's anatomy is predicted to change over time. Physician input can be used to generate or modify the virtual simulation(s). The present technology can generate one or more post-treatment virtual simulations based on the received physician input for review by the healthcare provider, patient, etc.

In some embodiments, the present technology can also predict, model, and/or simulate disease progression based on one or more potential surgical plans. For example, the disease progression module 120 may simulate what a patient's anatomy and/or spinal metrics may be 1, 2, 5, or 10 years post-surgery for several different surgical plans. The simulations may also incorporate non-surgical factors, such as patient age, height, weight, sex, activity level, other health conditions, or the like, as previously described. The system and/or a surgeon can use the disease progression to aid in selecting which surgical plan provides the best long-term efficacy, as described below. These simulations can also be used to determine patient-specific corrections that compensate for the projected diseases progression.

Accordingly, in some embodiments, multiple disease progression models (e.g., two, three, four, five, six, or more) are simulated to provide disease progression data for several different surgical plans. For example, the disease progression module can generate models that predict post-surgical disease progression for each of three different surgical plans. A surgeon or other healthcare provider can review the disease progression models and, based on the review, select which of the three surgical plans is likely to provide the patient with the best long-term outcome.

Based off of the modeled disease progression, the systems and methods described herein can also (i) identify a recommended time for surgical intervention, and/or (ii) identify a recommended type of surgical procedure for the patient. In some embodiments, the present technology therefore includes an intervention timing module 121 that includes an algorithm, machine learning model, or other software analytical tool for determining the optimal time for surgical intervention in a particular patient. This can be done, for example, by analyzing patient reference data that includes (i) pre-operative disease progression metrics for individual reference patients, (ii) disease metrics at the time of surgical intervention for individual reference patients, (iii) post-operative disease progression metrics for individual reference patients, and/or (iv) scored surgical outcomes for individual reference patients. The intervention timing module 121 can compare the disease metrics for a particular patient to the reference patient data sets to determine, for similar patients, the point of disease progression at which surgical intervention produced the most favorable outcomes.

As a non-limiting example, the reference patient data sets may include data associated with reference patients' sagittal vertical axis. The data can include (i) sagittal vertical axis values for individual patients over a period of time before surgical intervention (e.g., how fast and to what degree the sagittal vertical axis value changed), (ii) sagittal vertical axis of the individual patients at the time of surgical intervention, (iii) the change in sagittal vertical axis after surgical intervention, and (iv) the degree to which the surgical intervention was successful (e.g., based on pain, quality of life, or other factors). Based on the foregoing data, the intervention timing module 121 can, based on a particular patient's sagittal vertical axis value, identify at which point surgical intervention will have the highest likelihood of producing the most favorable outcome. Of course, the foregoing metric is provided by way of example only, and the intervention timing module 121 can incorporate other metrics (e.g., lumbar lordosis, pelvic tilt, sagittal vertical axis, cobb angel, coronal offset, disability scores, functional ability scores, flexibility scores, VAS pain scores) instead of or in combination with sagittal vertical axis to predict the time at which surgical intervention has the highest probability of providing a favorable outcome for the particular patient.

The intervention timing module 121 may also incorporate one or more mathematical rules based on value thresholds for various disease metrics. For example, the intervention timing module 121 may indicate surgical intervention is necessary if one or more disease metrics exceed a predetermined threshold or meet some other criteria. Representative thresholds that indicate surgical intervention may be necessary include SVA values greater than 7 mm, a mismatch between lumbar lordosis and pelvic incidence greater than 10 degrees, a cobb angle of greater than 10 degrees, and/or a combination of cobb angle and LL/PI mismatch greater than 20 degrees. Of course, other threshold values and metrics can be used; the foregoing are provided as examples only. In some embodiments, the foregoing rules can be tailored to specific patient populations (e.g., for males over 50 years of age, an SVA value greater than 7 mm indicates the need for surgical intervention). If a particular patient does not exceed the thresholds indicating surgical intervention is recommended, the intervention timing module 121 may provide an estimate for when the patient's metrics will exceed one or more thresholds, thereby providing the patient with an estimate of when surgical intervention may become recommended.

In some embodiments, the treatment planning module 118 identifies one or more types of surgical procedures for the patient based at least in part on the disease progression of the patient determined using the disease progression module 120 and/or the intervention timing module 121. The treatment planning module 118 may also incorporate one or more mathematical rules for identifying surgical procedures. As a non-limiting example, if a LL/PI mismatch is between 10 and 20 degrees, the treatment planning module 118 may recommend an anterior fusion surgery, but if the LL/PI mismatch is greater than 20 degrees, the treatment planning module may recommend both anterior and posterior fusion surgery. As another non-limiting example, if a SVA value is between 7mm and 15 mm, the treatment planning module may recommend posterior fusion surgery, but if the SVA is above 15 mm, the treatment planning module may recommend both posterior fusion surgery and anterior fusion surgery. Of course, other rules can be used; the foregoing are provided as examples only.

Without being bound by theory, incorporating disease progression modeling into the patient-specific surgical plans described herein may even further increase the effectiveness of the procedures and/or provide a surgeon more data by which to evaluate various surgical plans. For example, in many cases it may be disadvantageous to operate after a patient's disease progresses to an irreversible or unstable state. However, it may also be disadvantageous to operate too early, such as before the patient's disease is causing symptoms and/or if the patient's disease may not progress further. The disease progression module 120 and/or the intervention timing module 121 can therefore help identify the window of time during which surgical intervention in a particular patient has the highest probability of providing a favorable outcome for the patient.

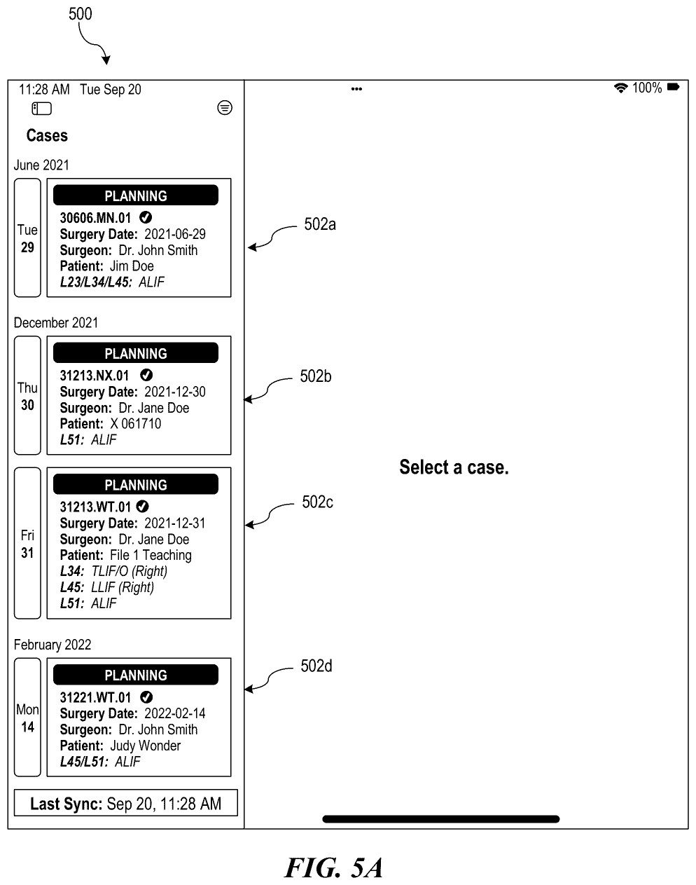

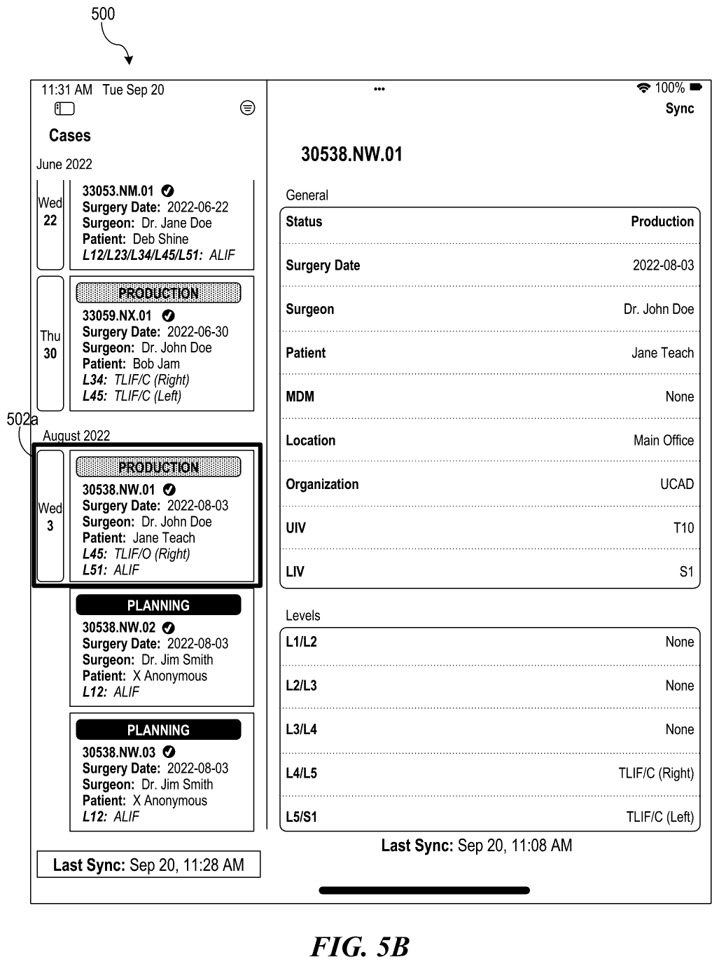

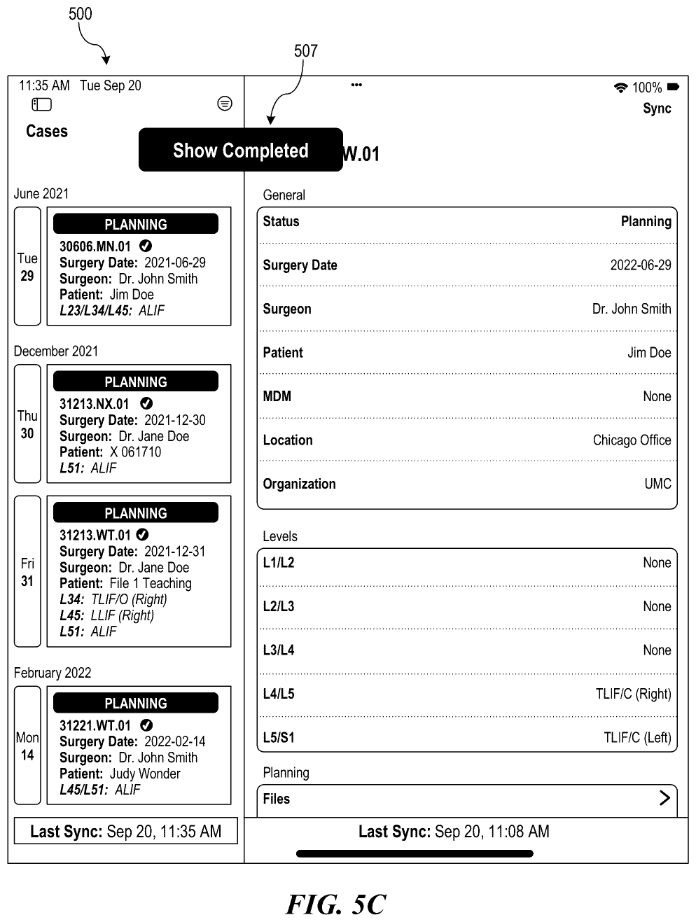

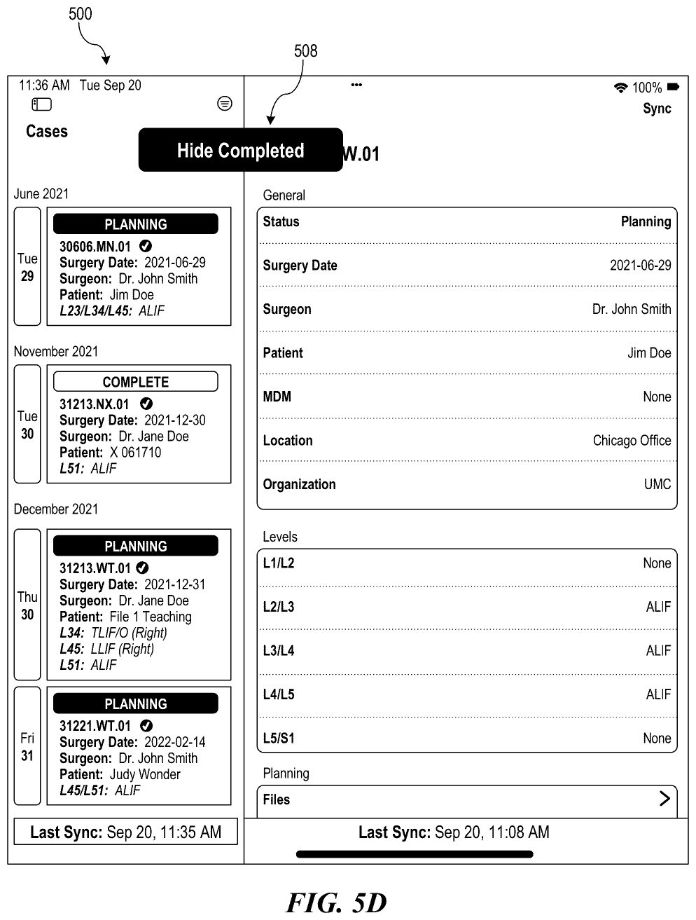

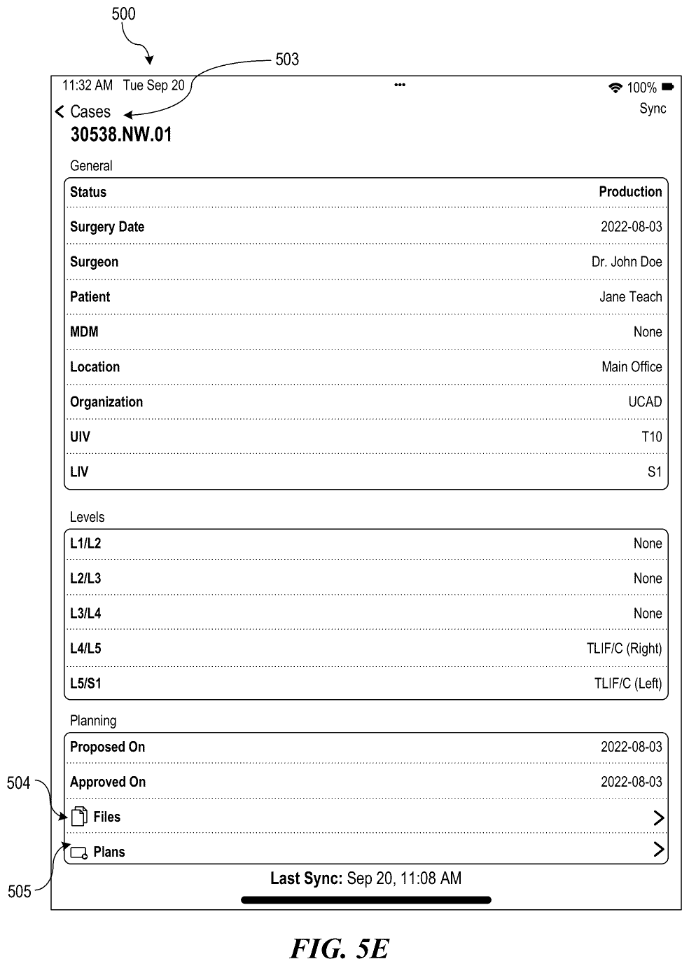

The surgical plan(s) generated by the treatment planning module 118 can be transmitted via the communication network 104 to the client computing device 102 for output to a user (e.g., clinician, surgeon, healthcare provider, patient). In some embodiments, the client computing device 102 includes or is operably coupled to a display 122 for outputting the treatment plan(s). The display 122 can include a graphical user interface (GUI) for visually depicting various aspects of the surgical plan(s). For example, the display 122 can show various aspects of a surgical procedure to be performed on the patient, such as the surgical approach, treatment levels, corrective maneuvers, tissue resection, and/or implant placement. To facilitate visualization, the surgical plan can include one or more virtual models of the surgical procedure that can be displayed via the display 122. For example, the surgical plan can include a first virtual model of pre-operative patient anatomy, a second virtual model of predicted post-operative patient anatomy if the surgical plan were to be executed, and/or a third virtual model comparing (e.g., overlaying) the predicted post-operative patient anatomy and the pre-operative patient anatomy. The display 122 may also display additional aspects of the surgical plan, such as predicted post-operative patient metrics, predicted disease progression metrics associated with the identified surgical procedure, etc. As another example, the display 122 can show a design for a medical device to be implanted in the patient in accordance with the transmitted surgical plan, such as a two- or three-dimensional model of the device design. The display 122 can also show patient information, such as two- or three-dimensional images or models of the patient's anatomy where the surgical procedure is to be performed and/or where the device is to be implanted. Examples of displays such as the display 122 depicting a surgical plan is provided below at FIGS. 5A-12. The client computing device 102 can further include one or more user input devices (not shown) allowing the user to modify, select, approve, and/or reject the displayed treatment plan(s).

In some embodiments, one or more aspects of the surgical plan are displayed using the surgical plan review program 125. For example, the review program 125, which may be implemented as a mobile phone application, a computer application, or the like, can display (e.g., via the display 122) one or more aspects of the surgical plan (e.g., a surgical procedure, a virtual model of patient anatomy, an implant, etc.). The review program 125 may provide an interactive interface that further enables a surgeon to select between different patients, select between different surgical plans for the same patient, compare surgical plans for the same patient, review the status of a surgical plan, provide feedback on a proposed surgical plan, accept a surgical plan, reject a surgical plan, etc. The review program 125 may further enable a surgeon or other user to select between different views of a virtual model of patient anatomy, and/or different views of a patient-specific implant to be used in the surgical plan. Additional details regarding features of the review program 125 are described below with reference to FIGS. 5A-12.

In some embodiments, the medical device design(s) generated by the treatment planning module 118 can be transmitted from the client computing device 102 and/or server 106 to a manufacturing system 124 for manufacturing a corresponding medical device. The manufacturing system 124 can be located on site or off site. On-site manufacturing can reduce the number of sessions with a patient and/or the time to be able to perform the surgery whereas off-site manufacturing can be useful make the complex devices. Off-site manufacturing facilities can have specialized manufacturing equipment. In some embodiments, more complicated device components can be manufactured off site, while simpler device components can be manufactured on site.

Various types of manufacturing systems are suitable for use in accordance with the embodiments herein. For example, the manufacturing system 124 can be configured for additive manufacturing, such as three-dimensional (3D) printing, stereolithography (SLA), digital light processing (DLP), fused deposition modeling (FDM), selective laser sintering (SLS), selective laser melting (SLM), selective heat sintering (SHM), electronic beam melting (EBM), laminated object manufacturing (LOM), powder bed printing (PP), thermoplastic printing, direct material deposition (DMD), inkjet photo resin printing, or like technologies, or combination thereof. Alternatively or in combination, the manufacturing system 124 can be configured for subtractive (traditional) manufacturing, such as CNC machining, electrical discharge machining (EDM), grinding, laser cutting, water jet machining, manual machining (e.g., milling, lathe/turning), or like technologies, or combinations thereof. The manufacturing system 124 can manufacture one or more patient-specific medical devices based on fabrication instructions or data (e.g., CAD data, 3D data, digital blueprints, stereolithography data, or other data suitable for the various manufacturing technologies described herein). Different components of the system 100 can generate at least a portion of the manufacturing data used by the manufacturing system 124. The manufacturing data can include, without limitation, fabrication instructions (e.g., programs executable by additive manufacturing equipment, subtractive manufacturing equipment, etc.), 3D data, CAD data (e.g., CAD files), CAM data (e.g., CAM files), path data (e.g., print head paths, tool paths, etc.), material data, tolerance data, surface finish data (e.g., surface roughness data), regulatory data (e.g., FDA requirements, reimbursement data, etc.), or the like. The manufacturing system 124 can analyze the manufacturability of the implant design based on the received manufacturing data. The implant design can be finalized by altering geometries, surfaces, etc. and then generating manufacturing instructions. In some embodiments, the server 106 generates at least a portion of the manufacturing data, which is transmitted to the manufacturing system 124.

The manufacturing system 124 can generate CAM data, print data (e.g., powder bed print data, thermoplastic print data, photo resin data, etc.), or the like and can include additive manufacturing equipment, subtractive manufacturing equipment, thermal processing equipment, or the like. The additive manufacturing equipment can be 3D printers, stereolithography devices, digital light processing devices, fused deposition modeling devices, selective laser sintering devices, selective laser melting devices, electronic beam melting devices, laminated object manufacturing devices, powder bed printers, thermoplastic printers, direct material deposition devices, or inkjet photo resin printers, or like technologies. The subtractive manufacturing equipment can be CNC machines, electrical discharge machines, grinders, laser cutters, water jet machines, manual machines (e.g., milling machines, lathes, etc.), or like technologies. Both additive and subtractive techniques can be used to produce implants with complex geometries, surface finishes, material properties, etc. The generated fabrication instructions can be configured to cause the manufacturing system 124 to manufacture the patient-specific orthopedic implant that matches or is therapeutically the same as the patient-specific design. In some embodiments, the patient-specific medical device can include features, materials, and designs shared across designs to simplify manufacturing. For example, deployable patient-specific medical devices for different patients can have similar internal deployment mechanisms but have different deployed configurations. In some embodiments, the components of the patient-specific medical devices are selected from a set of available pre-fabricated components and the selected pre-fabricated components can be modified based on the fabrication instructions or data.

The surgical plans described herein can be performed by a surgeon, a surgical robot, or a combination thereof, thus allowing for treatment flexibility. In some embodiments, the surgical procedure can be performed entirely by a surgeon, entirely by a surgical robot, or a combination thereof. For example, one step of a surgical procedure can be manually performed by a surgeon and another step of the procedure can be performed by a surgical robot. In some embodiments the treatment planning module 118 generates control instructions configured to cause a surgical robot (e.g., robotic surgery systems, navigation systems, etc.) to partially or fully perform a surgical procedure. The control instructions can be transmitted to the robotic apparatus by the client computing device 102 and/or the server 106.

Following the treatment of the patient in accordance with the surgical plan, treatment progress can be monitored over one or more time periods to update the data analysis module 116, treatment planning module 118, disease progression module 120, and/or intervention timing module 121. Post-treatment data can be added to the reference data stored in the database 110. The post-treatment data can be used to train machine learning models for developing patient-specific treatment plans, patient-specific medical devices, or combinations thereof.

It shall be appreciated that the components of the system 100 can be configured in many different ways. For example, in alternative embodiments, the database 110, the data analysis module 116, the treatment planning module 118, the disease progression module 120, and/or the intervention timing module 121 can be components of the client computing device 102, rather than the server 106. As another example, the database 110, the data analysis module 116, the treatment planning module 118, the disease progression module 120, and/or the intervention timing module 121 can be located across a plurality of different servers, computing systems, or other types of cloud-computing resources, rather than at a single server 106 or client computing device 102.

Additionally, in some embodiments, the system 100 can be operational with numerous other computing system environments or configurations. Examples of computing systems, environments, and/or configurations that may be suitable for use with the technology include, but are not limited to, personal computers, server computers, handheld or laptop devices, cellular telephones, wearable electronics, tablet devices, multiprocessor systems, microprocessor-based systems, programmable consumer electronics, network PCs, minicomputers, mainframe computers, distributed computing environments that include any of the above systems or devices, or the like.

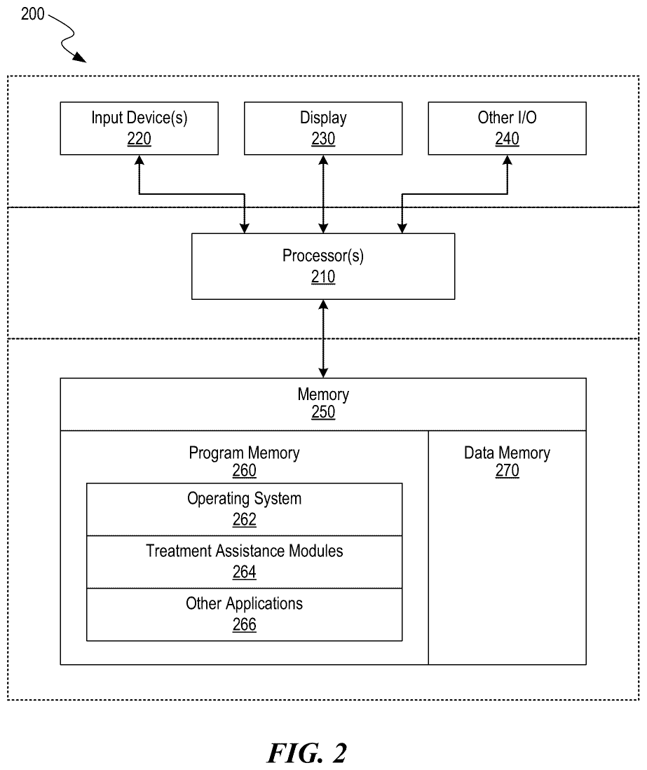

FIG. 2 illustrates a computing device 200 suitable for use in connection with the system 100 of FIG. 1, according to an embodiment. The computing device 200 can be incorporated in various components of the system 100 of FIG. 1, such as the client computing device 102 or the server 106. The computing device 200 includes one or more processors 210 (e.g., CPU(s), GPU(s), HPU(s), etc.). The processor(s) 210 can be a single processing unit or multiple processing units in a device or distributed across multiple devices. The processor(s) 210 can be coupled to other hardware devices, for example, with the use of a bus, such as a PCI bus or SCSI bus. The processor(s) 210 can be configured to execute one more computer-readable program instructions, such as program instructions to carry out of any of the methods described herein.

The computing device 200 can include one or more input devices 220 that provide input to the processor(s) 210, e.g., to notify it of actions from a user of the device 200. The actions can be mediated by a hardware controller that interprets the signals received from the input device and communicates the information to the processor(s) 210 using a communication protocol. Input device(s) 220 can include, for example, a mouse, a keyboard, a touchscreen, an infrared sensor, a touchpad, a wearable input device, a camera- or image-based input device, a microphone, or other user input devices.

The computing device 200 can include a display 230 used to display various types of output, such as text, models, virtual procedures, surgical plans, implants, graphics, and/or images (e.g., images with voxels indicating radiodensity units or Hounsfield units representing the density of the tissue at a location). In some embodiments, the display 230 provides graphical and textual visual feedback to a user. The processor(s) 210 can communicate with the display 230 via a hardware controller for devices. In some embodiments, the display 230 includes the input device(s) 220 as part of the display 230, such as when the input device(s) 220 include a touchscreen or is equipped with an eye direction monitoring system. In alternative embodiments, the display 230 is separate from the input device(s) 220. Examples of display devices include an LCD display screen, an LED display screen, a projected, holographic, or augmented reality display (e.g., a heads-up display device or a head-mounted device), and so on.

Optionally, other I/O devices 240 can also be coupled to the processor(s) 210, such as a network card, video card, audio card, USB, firewire or other external device, camera, printer, speakers, CD-ROM drive, DVD drive, disk drive, or Blu-Ray device. Other I/O devices 240 can also include input ports for information from directly connected medical equipment such as imaging apparatuses, including MRI machines, X-Ray machines, CT machines, etc. Other I/O devices 240 can further include input ports for receiving data from these types of machine from other sources, such as across a network or from previously captured data, for example, stored in a database.

In some embodiments, the computing device 200 also includes a communication device (not shown) capable of communicating wirelessly or wire-based with a network node. The communication device can communicate with another device or a server through a network using, for example, TCP/IP protocols. The computing device 200 can utilize the communication device to distribute operations across multiple network devices, including imaging equipment, manufacturing equipment, etc.

The computing device 200 can include memory 250, which can be in a single device or distributed across multiple devices. Memory 250 includes one or more of various hardware devices for volatile and non-volatile storage, and can include both read-only and writable memory. For example, a memory can comprise random access memory (RAM), various caches, CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory. In some embodiments, the memory 250 is a non-transitory computer-readable storage medium that stores, for example, programs, software, data, or the like. In some embodiments, memory 250 can include program memory 260 that stores programs and software, such as an operating system 262, one or more treatment assistance modules 264, and other application programs 266. The treatment assistance module(s) 264 can include one or more modules configured to perform the various methods described herein (e.g., the data analysis module 116 and/or treatment planning module 118 described with respect to FIG. 1). Memory 250 can also include data memory 270 that can include, e.g., reference data, configuration data, settings, user options or preferences, etc., which can be provided to the program memory 260 or any other element of the computing device 200.

### B. Select Methods of Modeling and Designing Patient-Specific Surgical Plans

The present technology includes systems and methods for generating a one or more patient-specific surgical plans, and transmitting the one or more patient-specific surgical plans to a surgeon or other healthcare provider for review, feedback, modification, and/or approval. As described below, the patient-specific surgical plans can include, among other things, a surgical procedure and a predicted post-operative outcome for the patient if the surgical procedure were to be performed.

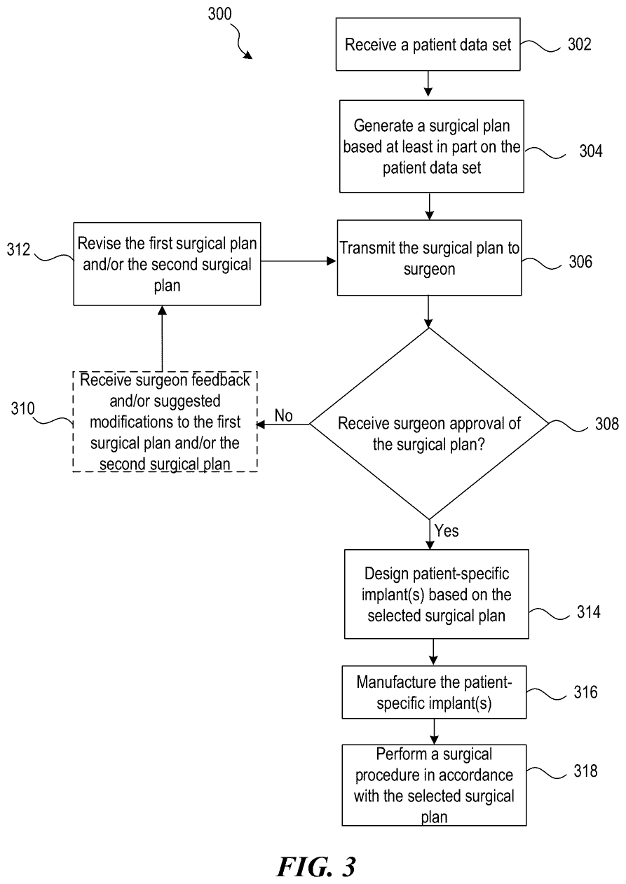

FIG. 3 is a flow diagram illustrating a method 300 for providing patient-specific medical care, according to an embodiment of the present technology. Some or all of the method 300 can be performed by various computing systems or software modules, including, for example, the computing systems described above with respect to FIGS. 1 and 2.

The method 300 can begin at block 302 by receiving a patient data set for a particular patient in need of medical treatment. The patient data set can include data representative of the patient's condition, anatomy, pathology, symptoms, medical history, preferences, and/or any other information or parameters relevant to the patient. For example, the patient data set can include surgical intervention data, treatment outcome data, progress data (e.g., surgeon notes), patient feedback (e.g., feedback acquired using quality of life questionnaires, surveys), clinical data, patient information (e.g., demographics, sex, age, height, weight, type of pathology, occupation, activity level, tissue information, health rating, comorbidities, health related quality of life (HRQL)), vital signs, diagnostic results, medication information, allergies, diagnostic equipment information (e.g., manufacturer, model number, specifications, user-selected settings/configurations, etc.) or the like. The patient data set can also include image data, such as camera images, Magnetic Resonance Imaging (MRI) images, ultrasound images, Computerized Aided Tomography (CAT) scan images, Positron Emission Tomography (PET) images, X-Ray images, and the like. In some embodiments, the patient data set includes data representing one or more of patient identification number (ID), age, gender, body mass index (BMI), lumbar lordosis, Cobb angle(s), pelvic incidence, disc height, segment flexibility, bone quality, rotational displacement, and/or treatment level of the spine. The patient data set can be received at a server, computing device, or other computing system. For example, in some embodiments the patient data set can be received by the server 106 shown in FIG. 1. In some embodiments, the computing system that receives the patient data set at block 302 also stores one or more software modules (e.g., the data analysis module 116, the treatment planning module 118, the disease progression module 120, and/or the intervention timing module 121, shown in FIG. 1, or additional software modules for performing various operations of the method 300).

In some embodiments, the received patient data set can include disease metrics such as lumbar lordosis, Cobb angles, coronal parameters (e.g., coronal balance, global coronal balance, coronal pelvic tilt, etc.), sagittal parameters (e.g., pelvic incidence, sacral slope, thoracic kyphosis, etc.) and/or pelvic parameters. The disease metrics can include micro-measurements (e.g., metrics associated with specific or individual segments of the patient's spine) and/or macro-measurements (e.g., metrics associated with multiple segments of the patient's spine). In some embodiments, the disease metrics are not included in the patient data set, and the method 300 includes determining (e.g., automatically determining) one or more of the disease metrics based on the patient image data or associated virtual models, as described below. In some embodiments, the received patient data can include functional mobility test scores (e.g., step test, six-meter walk test, sit-to-stand test, timed up and go test, etc.). The received patient data set can include additional subjective test scores that reflect aspects of the patient condition, such as pain tests (e.g., Visual Analog Scale (VAS) pain scores, Low Back Pain Rating scale scores, etc.), disability tests (e.g., Oswestry Disability Index scores, Quebec back pain disability test scores, etc.), quality of life tests (e.g., Quality of Life Scale scores), etc.

The method 300 can continue at block 304 by generating a surgical plan based at least in part on the patient data set received at block 302. As described in detail below, the surgical plan can include a target location or region of interest for surgical intervention and one or more surgical procedures or interventions to be performed at the region of interest. The surgical plan can also include predicted post-operative data associated with performing the surgical procedure at the target location. For example, the surgical plan may include a predicted or target post-operative anatomical configuration shown as a two or three dimensional virtual model. In some embodiments, the surgical plan also includes additional predicted post-operative analytics, such as predicted disease progression, predicted patient satisfaction, predicted patient mobility, predicted patient pain, predicted patient quality of life, etc.

In some embodiments, the operation of generating the surgical plan includes identifying a specific target location to be involved in the surgical procedure. For example, in the context of spinal surgery, generating the surgical plan may include identifying one or more vertebral levels for surgical intervention. In some embodiments, the vertebral level is a cervical vertebral level (e.g., C1-C5), a thoracic vertebral level (e.g., T1-T12), a lumbar vertebral level (e.g., L1-L5), and/or the sacrum. In some embodiments, the identified target location includes a specific range of vertebral levels to be involved in a surgery (e.g., L1-L3, L3-L5, L4-T12, C1-C3, etc.). The identified target location may include two, three, four, five, or more vertebral levels. Of course, the foregoing target locations are provided by way of example only, and the present technology is not limited to the anatomical locations listed above. Indeed, in some embodiments the target location may include anatomical structures other than the spine, such as the hip, knee, ankle, shoulder, elbow, wrist, hand, the jaw, the skull, or other anatomical locations, as described throughout this Detailed Description.

The target location can be identified by reviewing image data of the patient. In some embodiments, a computing system (e.g., the computing system 106 of FIG. 1) and/or one or more software modules (e.g., the treatment planning module 118 of FIG. 1) can review and analyze patient image data and automatically identify the target location. In such embodiments, a trained machine learning program or other software-based program can analyze patient image data, extract measurements from the patient image data, compare the extracted measurements to reference data (e.g., predetermined thresholds or ranges associated with “healthy” patients normalized for age, sex, gender, etc.), and identify anatomical regions that are candidates for surgical correction. Alternatively or additionally, the target location can be identified and/or confirmed through other suitable means, such as via a technician or healthcare provider reviewing image data and identifying anatomical deformities.

As provided above, in some embodiments the operation of generating the surgical plan also includes identifying a surgical procedure for the patient. In embodiments in which the surgical plan includes identifying a target location, the surgical procedure can be associated with the target location. In the context of spinal surgery, representative surgical procedures include spinal fusion, artificial disc replacement, vertebroplasty, kyphoplasty, spinal laminectomy/decompression, discectomy, facetectomy, foraminotomy, or other spine surgery procedures. Examples of spinal fusion surgery include posterior lumbar interbody fusion (PLIF), anterior lumbar interbody fusion (ALIF), transverse or transforaminal lumbar interbody fusion (TLIF), lateral lumbar interbody fusion (LLIF), direct lateral lumbar interbody fusion (DLIF), or extreme lateral lumbar interbody fusion (XLIF). The foregoing are provided by way of example only, and the present technology can include identifying any type of spinal or other surgical procedures at block 304a.

The surgical procedure associated with the surgical plan can be identified using any of the methods and systems described herein. For example, in some embodiments the computing system 106 of FIG. 1 and/or the associated software modules (e.g., the treatment planning module 118) can identify one or more surgical procedures based on, for example, user input, the received or extracted patient data, and/or the identified target location(s). For example, if the computing system 106 determines that a patient is suffering from disc degeneration from L3-L5, the computing system 106 may recommend a PLIF procedure to fuse L2-T12. Alternatively, the computing system 106 may recommend an artificial disc replacement at L3-L4 and L4-L5 to correct the degeneration while preserving motion. The surgical procedure can be identified using other methods and systems as well.

In some embodiments, the operation at block 304 can include reviewing and/or analyzing multiple types of surgical procedures and/or surgical steps to identify the surgical procedure for inclusion within the surgical plan. Types of surgical procedures and/or surgical steps can be selected for inclusion with the surgical plan (or eliminated from inclusion with the surgical plan) based on, for example, user input, insurance coverage of the procedure or step, healthcare provider parameters (e.g., based on healthcare provider ranking/scores such as hospital/physician expertise, number of similar procedures performed, hospital ranking for procedure, etc.), healthcare resource parameters (e.g., diagnostic equipment, facilities, surgical equipment such as surgical robots), and/or other non-patient related information (e.g., information that can be used to score, predict outcomes and risk profiles for procedures for the present healthcare provider, and/or rank procedures).

In some embodiments, the operation of generating the surgical plan includes identifying or designing a corrected anatomical configuration for the patient (the corrected anatomical configuration can also be referred to herein as the “planned configuration,” “optimized geometry,” “post-operative anatomical configuration,” or “target outcome”). The corrected anatomical configuration can reflect the desired and/or predicted anatomy of the patient if the surgical plan were performed. In some embodiments, generating the surgical plan includes generating one or more virtual models (two-dimensional models, three-dimensional models, etc.) showing the corrected anatomical configuration. The virtual model may include some or all of the patient's anatomy within the target location (e.g., any combination of tissue types including, but not limited to, bony structures, cartilage, soft tissue, vascular tissue, nervous tissue, etc.). In some embodiments, the virtual models can be multi-dimensional (e.g., two-dimensional or three-dimensional) virtual models and can include, for example, CAD data, material data, surface modeling, manufacturing data, or the like. The CAD data can include, for example, solid modeling data (e.g., part files, assembly files, libraries, part/object identifiers, etc.), model geometry, object representations, parametric data, object representations, topology data, surface data, assembly data, metadata, etc. Examples of virtual models are described in U.S. application Ser. Nos. 16/048,167, 16/242,877, 16/207,116, 16/352,699, 16/383,215, 16/569,494, 16/699,447, 16/735,222, 16/987,113, 16/990,810, 17/085,564, 17/100,396, 17/342,329, 17/518,524, 17/531,417, 17/835,777, 17/851,487, 17/867,621, and 17/842,242, each of which is incorporated by reference herein in its entirety.

In some embodiments, one or more clinical checks can be performed on or using the generated virtual model showing predicted post-operative patient anatomy. Clinical checks can include measuring features (e.g., disc space height, lordosis, etc.) of the virtual model to determine whether an acceptable metric is achieved. The system can store acceptable metrics based on historical patient data or other accepted medical criteria. If one or more measured metrics fall outside an acceptable range, the user can be notified so that the user can determine whether the surgical plan should be updated or discarded. Clinical checks can be performed any number of times throughout the method 300 to ensure that individual steps of the surgical planning does not fall outside of acceptable criteria (e.g., clinical checks can be performed until the virtual model showing predicted post-operative anatomy conforms with the one or more metrics). In some embodiments, the corrected anatomical configuration is identified/determined before the surgical procedure and/or target location. That is, a computing system or user can model a preferred anatomical outcome, and, based on the desired anatomical outcome, identify a surgical procedure and target location that will achieve the desired anatomical outcome once performed.

In some embodiments, generating the surgical plan includes generating one or more patient metrics associated with the corrected anatomical configuration. In the context of spinal surgery, patient metrics may include, for example, coronal parameters, sagittal parameters, pelvic parameters, Cobb angles, shoulder tilt, iliolumbar angles, coronal balance, lordosis angles, intervertebral space height, or other similar spinal parameters. Similar as described above, the patient metrics can be determined before identifying a surgical procedure and/or target location for surgical intervention. That is, a computing system or user can use the patient metrics to identify a surgical procedure and target location that will achieve the patient metrics once performed.

The surgical plan can include additional features. In some embodiments, for example, the surgical plan can include predicted disease progression, predicted patient satisfaction, predicted patient mobility, predicted patient pain, predicted patient quality of life, or the like. For example, the surgical plan may include estimates of disease progression if the patient were to undergo the identified surgical procedure at the identified target location. That is, the surgical plan can include virtual models (e.g., two-dimensional or three-dimensional virtual models) of patient anatomy at various intervals post-operation. For example, the surgical plan may include a predictive model of patient anatomy at one or more of 6 months post-op, 1 year post-op, 2 years post-op, 3 years post-op, 4 year post-op, 5 years post-op, 6 years post-op, 7 years post-op, 8 years post-op, 9 years post-op, and/or 10 years post-op. The disease progression model may also include predicted patient metrics (e.g., any of the patient metrics described herein, including coronal parameters, sagittal parameters, pelvic parameters, Cobb angles, shoulder tilt, iliolumbar angles, coronal balance, lordosis angles, intervertebral space height, or other similar spinal parameters) at any of the various post-operative intervals identified above, in addition to or in lieu of including the virtual model of predicted patient anatomy.

Once generated, the surgical plan can be digitally displayed as a surgical report on one or more display screens for ease of review, editing, annotation, the like. In some embodiments, the surgical plan can be stored as computer-executable instructions that can be executed via the surgical plan review module 123 on the client computing device 102 of FIG. 1. More specifically, the surgical plan can be reviewed using the surgical plan review program 125, shown in FIG. 1 and described in greater detail below with reference to FIGS. 5A-12.

In some embodiments, the operation of generating the surgical plan at block 304 includes generating a plurality of candidate surgical plans (or subsets of surgical plans such as surgical procedures), and then selecting the surgical from within the plurality of candidate surgical plans. For example, in some embodiments a computing system can automatically identify a plurality (e.g., two, three, four, five, six, seven, eight, nine, ten, or more) of surgical plans (or subsets of surgical plans such as surgical procedures) based on the patient-data set and/or one or more user-inputted criteria. The identified candidate surgical plans may be ranked and/or scored based on various factors, including predicted patient outcomes, user-review, etc. The highest ranked identified candidate surgical plans (e.g., based on predicted patient outcomes) can be selected as the surgical plan. In some embodiments, certain ranked surgical plans may not be selected as the surgical plan based on user review and/or failure to meet various user criteria. For example, if a particular surgical plan is identified as requiring a surgical procedure that the physician is unfamiliar with, the particular surgical plan may not be selected, and the method can instead include selecting the next best surgical plan as the surgical plan. Accordingly, in some implementations, physician-specific scoring is used to score candidate procedures/surgical plans before selecting the surgical plan. For example, procedures with scores meeting a threshold score (e.g., threshold post-operative metrics score, physician inputted threshold score, threshold outcome score, etc.) can be identified for user review. The system can therefore compare advantages and disadvantages of candidate procedures with respect to each other before selecting the surgical plan.

Once the surgical plan is generated at block 304, the method 300 can continue at block 306 by transmitting the surgical plan to a surgeon. In some embodiments, the same computing system used at blocks 302 and 304 can transmit the surgical plan to a computing device for surgeon review (e.g., the client computing device 102 described in FIG. 1). This can include directly transmitting the surgical plan to the computing device or uploading the first and second surgical plans to a cloud or other storage system for subsequent downloading.

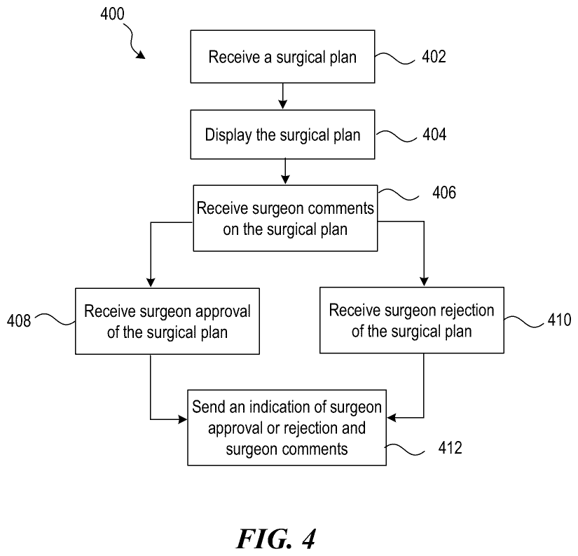

The surgeon can review the surgical plan and, at block 308, approve or disapprove of the surgical plan. For example, the surgeon may review the surgical plan using the surgical plan reviewing program 125 (FIG. 1) to determine whether the surgeon deems the surgical plan acceptable. This may include, for example, reviewing the surgical plans target locations, surgical procedure, target/predicted post-operative anatomical configuration, and predicted post-operative patient metrics. Additional aspects of using the surgical plan reviewing program 125 to review the surgical plan are described in Section C below with reference to FIGS. 4-12.

In some embodiments, the surgeon may not approve the surgical plan at block 308. In such embodiments, the surgeon can optionally provide feedback and/or suggested modifications to the surgical plan (e.g., by adjusting the virtual model or changing one or more aspects about the plan, providing comments on or more requested changes to the surgical plan, etc.). Accordingly, the method 300 can optionally include receiving (e.g., via the computing system) the surgeon feedback and/or suggested modifications at block 310. This may include, for example, modifying target locations for surgical intervention, surgical procedures, and/or target post-operative anatomical configuration. If surgeon feedback and/or suggested modifications are received at block 310, the method 300 can continue at block 312 by revising (e.g., automatically revising via the computing system) the surgical plan based at least in part on the surgeon feedback and/or suggested modifications received at block 310. In some embodiments, the surgeon does not provide feedback and/or suggested modifications if they reject the surgical plan. In such embodiments, block 310 can be omitted, and the method 300 can continue at block 312 by revising (e.g., automatically revising via the computing system) the surgical plans by selecting new and/or additional reference patient data sets and/or generating a new candidate surgical plan. The revised and/or new surgical plan can then be transmitted to the surgeon for review.

The operations at blocks 306, 308, 310, and 312 can be repeated as many times as necessary until the surgeon selects and approves a particular surgical plan. In some embodiments, the method 300 can include performing one or more checks (e.g., clinical checks, manufacturability checks, etc.) on the one or more suggested modifications provided by the surgeon before revising the surgical plan based on the suggested modifications. Similar checks can also be performed on an approved surgical plan.

Once surgeon approval of a surgical plan is received at block 308, the method 300 can continue at block 314 by designing (e.g., via the same computing system that performed blocks 302-308) one or more patient-specific implants based on the selected surgical plan. For example, the patient-specific implant can be designed based on the target location and surgical procedure included in the selected surgical plan. The patient-specific implant(s) can also be specifically designed such that, when implanted in the particular patient at the target location using the identified surgical procedure, it directs the patient's anatomy to occupy the target post-operative anatomical configuration (e.g., transforming the patient's anatomy from the patient's native anatomical configuration to the corrected anatomical configuration). The patient-specific implant can be designed such that, when implanted, it causes the patient's anatomy to occupy the corrected anatomical configuration for the expected service life of the implant (e.g., 5 years or more, 10 years or more, 20 years or more, 50 years or more, etc.). In some embodiments, the patient-specific implant is designed solely based on the virtual model of the corrected anatomical configuration and/or without reference to pre-operative patient images.

The patient-specific implant can be any of the implants described herein or in any patent references incorporated by reference herein. For example, the patient-specific implant can include one or more of screws (e.g., bone screws, spinal screws, pedicle screws, facet screws), interbody implant devices (e.g., intervertebral implants), cages, plates, rods, discs, fusion devices, spacers, rods, expandable devices, stents, brackets, ties, scaffolds, fixation device, anchors, nuts, bolts, rivets, connectors, tethers, fasteners, joint replacements (e.g., artificial discs), hip implants, or the like. A patient-specific implant design can include data representing one or more of physical properties (e.g., size, shape, volume, material, mass, weight), mechanical properties (e.g., stiffness, strength, modulus, hardness), and/or biological properties (e.g., osteo-integration, cellular adhesion, anti-bacterial properties, anti-viral properties) of the implant. For example, a design for an orthopedic implant can include implant shape, size, material, and/or effective stiffness (e.g., lattice density, number of struts, location of struts, etc.). Examples of patient-specific implants that can be designed at block 314 are described in U.S. application Ser. Nos. 16/048,167, 16/242,877, 16/207,116, 16/352,699, 16/383,215, 16/569,494, 16/699,447, 16/735,222, 16/987,113, 16/990,810, 17/085,564, 17/100,396, 17/342,329, 17/518,524, 17/531,417, 17/835,777, 17/851,487, 17/867,621, and 17/842,242, each of which is incorporated by reference herein in its entirety.

In some embodiments, designing the implant at block 316 can optionally include generating fabrication instructions for manufacturing the implant. For example, the computing system may generate computer-executable fabrication instructions that that, when executed by a manufacturing system, cause the manufacturing system to manufacture the implant.

In some embodiments, the patient-specific implant is designed at block 316 only after the surgeon has selected a surgical plan. Accordingly, in some embodiments, the implant design is neither transmitted to the surgeon with the surgical plan at block 308, nor manufactured before receiving surgeon approval of the surgical plan. Without being bound by theory, waiting to design the patient-specific implant until after the surgeon approves the surgical plan may increase the efficiency of the method 300 and/or reduce the resources necessary to perform the method 300. In other embodiments, one or more patient-specific implants can be designed and included in the surgical plans transmitted to the surgeon at block 306. Accordingly, in some embodiments the operation at block 314 can be included within the block 304.

The method 300 can continue at block 316 by manufacturing the patient-specific implant. The implant can be manufactured using additive manufacturing techniques, such as 3D printing, stereolithography, digital light processing, fused deposition modeling, selective laser sintering, selective laser melting, electronic beam melting, laminated object manufacturing, powder bed printing, thermoplastic printing, direct material deposition, or inkjet photo resin printing, or like technologies, or combination thereof. Alternatively or additionally, the implant can be manufactured using subtractive manufacturing techniques, such as CNC machining, electrical discharge machining (EDM), grinding, laser cutting, water jet machining, manual machining (e.g., milling, lathe/turning), or like technologies, or combinations thereof. The implant may be manufactured by any suitable manufacturing system (e.g., the manufacturing system 124 shown in FIG. 1). In some embodiments, the implant is manufactured by the manufacturing system executing the computer-readable fabrication instructions generated by the computing system at block 316.

Once the implant is manufactured at block 316, the method 300 can continue at block 318 by performing the selected surgical plan and implanting the patient-specific implant into the patient. Aspects of the surgical plan, such as some or all of the surgical procedure, can be performed manually, by a robotic surgical platform (e.g., a surgical robot), or a combination thereof. In embodiments in which the surgical procedure is performed at least in part by a robotic surgical platform, the surgical plan can include computer-readable control instructions configured to cause the surgical robot to perform, at least partly, the patient-specific surgical procedure.

The method 300 can be implemented and performed in various ways. In some embodiments, the operations at blocks 302-314 can be performed by a computing system associated with a first entity, block 316 can be performed by a manufacturing system associated with a second entity, and block 318 can be performed by a surgical provider, surgeon, and/or robotic surgical platform associated with a third entity. Any of the foregoing blocks may also be implemented as computer-readable instructions stored in memory and executable by one or more processors of the associated computing system(s).

### C. Select Embodiments of Surgical Plan Review Programs and Associated Methods

FIG. 4 is a flowchart of a method 400 of providing patient specific medical care, according to embodiments of the present technology. More specifically, the method 400 is directed to reviewing, modifying, and/or approving a candidate patient-specific surgical plan using a surgical plan review program, such as the surgical plan described with reference to the method 300 of FIG. 3 and the surgical plan review program 125 of FIG. 1, respectively. Accordingly, the method 400 can be a computer-method performed via a client computing device, such as a computer, tablet, phone, or the like (e.g., the computing device 102 of FIG. 1).

The method 400 can begin at block 402 be receiving a surgical plan at the client computing device. In some embodiments, the surgical plan is received at the client computing device by downloading the surgical plan from a server or cloud-based storage platform. In other embodiments, the surgical plan is directly received at the client computing device from another computing system (e.g., the system 106) via Wi-Fi, cellular, Bluetooth, NFC, or another communication network.

Once the surgical plan is received at block 402, the method 400 can continue at block 402 by displaying the surgical plan on the client computing device. In some embodiments, displaying the surgical plan includes launching the surgical plan reviewing program 125 and/or opening the surgical plan in the surgical plan review program 125. As described throughout this Detailed Description, the surgical plan review program 125 provides a convenient, user-friendly interface that enables a surgeon or other user to review, comment on, approve, and/or reject the surgical plan.

In some embodiments, displaying the surgical plan at block 404 includes displaying, via the display screen, the surgical plan's target location for surgical intervention, the surgical plan's surgical procedure, the surgical plan's target post-operative anatomical configuration (e.g., a virtual model of the target post-operative anatomical configuration), metrics associated with the target post-operative anatomical configuration, a comparison between metrics associated with current patient anatomy and metrics associated with the target post-operative anatomical configurations, disease progression predictions, patient outcome predictions, etc. In some embodiments, the display is interactive such that a user (e.g., a surgeon) can toggle between different aspects of the surgical plans, zoom in or out (e.g., on the models of target post-operative anatomical configuration), annotate the plans, provide feedback on the plans, etc. Examples of the interactivity of the surgical plans when viewed using the surgical plan review program 125, and features provided by the surgical plan review program 125, are described below with reference to FIGS. 5A-12.

The method 400 can continue at block 406 by receiving surgeon comments on the surgical plan. Surgeon comments may include, without limitation, requested changes to the surgical plan, such as a change to the target location for surgical intervention, the surgical procedure, the target post-operative anatomical configuration, patient metrics associated with the target post-operative anatomical configuration, implant designs, implant type, number of implants, etc. Surgeon comments may also include notes by the surgeon about the surgical plan, in lieu of or in addition to the requested changes to the surgical plan. Surgeon comments may further yet include questions about the surgical plan that can be transmitted back to the computing system for technician review. In some embodiments, a surgeon may not have any comments on the surgical plan, in which case the operation at block 406 can be omitted from the method 400.

The method 400 can continue by, at block 408, receiving surgeon approval of the surgical plan, or, at block 410, receiving surgeon rejection of the surgical plan. The method 400 can continue at block 412 by sending (a) an indication of whether the surgical plan was approved or rejected at blocks 408 and 410, and (b) any comments received from the surgeon at block 406. In some embodiments, this includes sending an indication to the computing system that originally transmitted surgical plan to the client computing device and/or uploaded the surgical plan to the server.

As described above, surgeon review of the surgical plan can be facilitated via a surgical plan review program 125, which can be a mobile phone application, computer application, etc. that is stored as instructions on the client computing device as a surgical plan review module 123. FIGS. 5A-12 provide additional details regarding the surgical plan review program 125.

The surgical plan review program 125 can include different dashboards for reviewing surgical plans. For examples, FIGS. 5A-5E are screenshots showing various aspects of a representative case display dashboard 500 of the surgical plan review program 125 in accordance with embodiments of the present technology. Referring first to FIG. 5A, which is a first screenshot of the case display dashboard 500, the case display dashboard 500 can display a plurality of patient cases 502a-d (collectively referred to as “the patient cases 502”). Each of the patient cases 502 can correspond to a different patient receiving care from the surgeon. As shown, the patient cases 502 displayed on the dashboard can include identifier information that enables a surgeon to distinguish between the cases. Suitable identifier information can include, but is not limited to, case number, surgery date, surgeon name, patient name, patient medical record number, surgical target, surgical type, stage of planning, or the like. In embodiments in which the surgical plan review program 125 is shared by a healthcare clinic or system, the patient cases 502 can correspond to patients receiving care from the healthcare clinic or system (e.g., not limited to patients receiving care from a specific surgeon). The case display dashboard 500 can display the patient cases 502 in chronological order by date of surgery, as shown in FIG. 5A. In other embodiments, the patient cases 502 may be displayed in alphabetical order, chronological order based on when the case was received, case status, or other suitable criteria. A user (e.g., the surgeon) can select a particular case for more detailed review from the dashboard 500. Although only four patient cases 502 are shown, the dashboard 500 can include all active cases for a particular surgeon or healthcare provider, and so the dashboard 500 may include more or fewer cases than shown in FIG. 5A. In some embodiments, the dashboard 500 may further include past (e.g., inactive) patient cases 502.

FIG. 5B is a second screenshot of the representative case display dashboard 500 of the surgical plan review program 125 with a particular patient case 502a selected from among the plurality of patient cases 502. As shown, once a particular patient case 502a is selected, additional details of the particular patient case 502a are displayed in the case display dashboard 500. For example, in the illustrated embodiment, additional details such as status (e.g., planning, production, post-operative, etc.) surgery date, surgeon name, patient name, patient medical record number, MDM, location, organization, treatment type, treatment levels, or the like can be displayed. Other information beyond those shown and described above can also be displayed. In some embodiments, the information may not simultaneously fit on a single screen, and the display can be a scrollable display so that a user can quickly and easily view additional simply by scrolling up or down on the page.

The case display dashboard can be modified or organized according to a user's preferences. For example, FIGS. 5C and 5D are third and fourth screenshots of the representative case display dashboard 500 of FIGS. 5A and 5B, illustrating additional features of the case display dashboard 500 in accordance with embodiments of the present technology. In particular, FIG. 5C provides a first view with completed cases hidden and with a third selector 507 (e.g., button) for showing completed cases, while FIG. 5D provides a second view with completed cases shown and with a fourth selector 508 for hiding completed cases. A user can therefore switch between the views shown in FIGS. 5C and 5D by selecting the corresponding third selector 507 or fourth selector 508. Although shown as selecting between completed and pending cases, in some embodiments the dashboard 500 can provide options for sorting the cases based on additional categories, such as sorting by a stage of the case (e.g., planning, production, pre-operative, post-operative, completed, etc.). The dashboard 500 can also be organized and sorted in other manners according to a user's preference.

FIG. 5E is a fifth screenshot of the case display dashboard 500 of the surgical plan review program 125, showing an additional embodiment of the case display dashboard 500 in accordance with embodiments of the present technology. The embodiment of FIG. 5E is generally similar to the embodiment of FIG. 5B, but does not display a thread of the patient cases 502 when the particular patient case 502a is selected. Rather, the particular patient case 502a is opened in a new screen, window, or tab. A user can nevertheless easily switch between patient cases 502 simply by selecting a return option 503 for returning to the home display, e.g., as shown in FIG. 5A. Similar to the embodiment shown in FIG. 5B, the embodiment in FIG. 5C also shows details associated with the surgical plan, including surgery date, surgeon name, patient name, patient medical record number, MDM, location, organization, treatment type, treatment levels. Other information beyond those shown and described above can also be displayed. The view of the dashboard 500 in FIG. 5C also provides a first selector 504 for viewing files associated with the patient case, and a second selector 505 for viewing the surgical plan for the patient case 502a. In some embodiments, the surgical plan review program 125 opens a surgical plan review dashboard to facilitate user review of the surgical plan in response to a user selecting the second selector 505, as described below.

As noted above, the surgical plan review program 125 also includes a surgical plan review dashboard that enables a surgeon or other user to review aspects of a proposed surgical plan. FIGS. 6A-12 illustrate various aspects of a surgical plan review dashboard 600 of the surgical plan review program 125 in accordance with embodiments of the present technology. As described in greater detail below, the surgical plan review dashboard 600 enables a surgeon or other user to review various aspects of a proposed patient-specific surgical plan, including pre-operative metrics, predicted post-operative metrics, virtual models of patient anatomy, and the like. In some embodiments, a user can launch the surgical plan review dashboard 600 by selecting the second selector 505 (FIG. 5E) for viewing the surgical plan from the case display dashboard 500 (FIGS. 5A-5E).

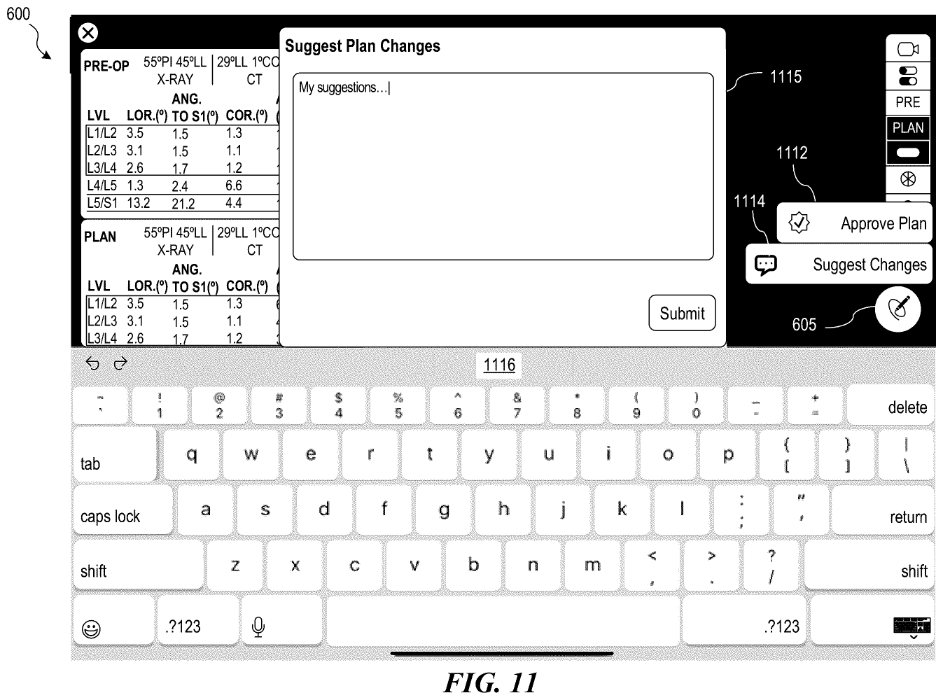

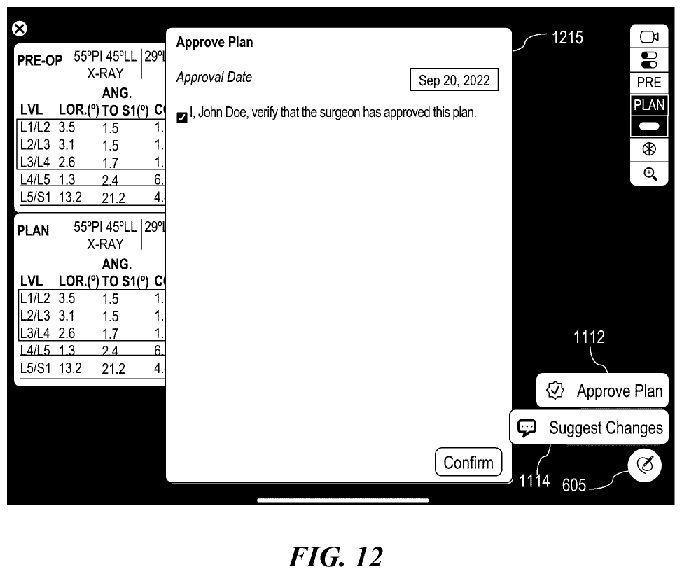

FIG. 6A is a first screenshot of the surgical plan review dashboard 600 illustrating various select features of the surgical plan review dashboard 600. As shown in FIG. 6A, the surgical plan review dashboard 600 includes a menu 630 with various selectors (e.g., virtual buttons, tabs, or the like) that a user can toggle on and off to selectively control the information displayed by the surgical plan review dashboard 600. For example, in FIG. 6A, a first selector 632 is toggled on/selected. As a result of the first selector 623 being “on”, the surgical plan review dashboard 600 displays a first table or chart 610 showing pre-operative patient metrics and a second table or chart 612 showing predicted post-operative patient metrics. As set forth above, the pre-operative patient metrics in the first table 610 are the patient's current metrics, and the post-operative patient metrics in the second table 612 are predicted patient metrics for the patient if the surgical plan is approved and performed by the surgeon. In the embodiment illustrated in FIG. 6A, a second selector 634 labeled “PRE” is also toggled on/selected from the menu 630. As a result of the second selector 634 being “on”, the surgical plan review dashboard 600 displays a pre-op virtual model 620 (which can also be referred to as a “first virtual model 620”) showing pre-operative patient anatomy. The pre-op virtual model 620 can be a two-dimensional or three-dimensional model of the patient's current (e.g., pre-operative or native) anatomy at a region of interest. The surgical plan review dashboard 600 further includes a plan feedback selector 605, which can be selected to open a window for commenting on and/or approving the displayed surgical plan, as described in detail with reference to FIGS. 11 and 12.

FIG. 6B is a second screenshot of the surgical plan review dashboard 600 illustrating additional features of the dashboard. More specifically, in FIG. 6B a third selector 635 labeled “PLAN” is toggled on/selected from the menu 630, in addition to the first selector 632. As a result of the third selector 635 being “on”, the surgical plan review dashboard 600 displays a surgical plan in the form of predicted post-op virtual model 622 (which can also be referred to as a “second virtual model 622”). Similar to the pre-op virtual model 620 in FIG. 6A, the post-op virtual model 622 can be a two-dimensional or three-dimensional virtual model of patient anatomy at a region of interest. However, unlike the pre-op virtual model 620, the post-op virtual model 622 illustrates predicted post-operative patient anatomy at the region of interest if the surgical plan were to be approved and performed by the surgeon. In some embodiments, the post-op virtual model 622 may further include one or more virtual implants that correspond to implants that would be implanted into the region of interest if the surgical plan were to be approved and performed by the surgeon. For example, in the illustrated embodiment a first virtual implant 626a and a second virtual implant 626b (collectively, “the virtual implants 626”) are shown between the L4-L5 vertebral bodies and the L5-S1 vertebral bodies, respectively. The foregoing are provided by way of example only—depending on the surgical plan, more or fewer virtual implants may be displayed in the post-op virtual model 622. Similarly, other types of virtual implants beyond interbody implants may be shown, depending on the type of surgical intervention called for in the surgical plan.

FIG. 6C is a third screenshot of the surgical plan review dashboard 600 illustrating further features of the surgical plan review dashboard. More specifically, in FIG. 6C both the second selector 634 (“PRE”) and the third selector 635 (“PLAN”) are toggled on/selected from the menu 630. As a result of both the second selector 634 and the third selector 635 being “on”, the surgical plan review dashboard 600 displays a comparative virtual model 624 (which can also be referred to as a “third virtual model 624”), which shows the post-op virtual model 622 (FIG. 6B) overlaying the pre-op virtual model 620 (FIG. 6A). In some embodiments, the comparative virtual model 624 is generated or built using a comparator of a design platform, such as the comparator described above with reference to the analysis module 116 of FIG. 1. The comparative virtual model 624 can therefore utilize one or more keying features to ensure that the pre-op virtual model 620 and the post-po virtual model 622 are aligned correctly. In some embodiments, the pre-op virtual model 620 and the post-op virtual model 622 are shown in different colors or shades to more easily compare current patient anatomy with predicted post-operative patient anatomy using the comparative virtual model 624.

In some embodiments, the surgical plan review dashboard can display or otherwise illustrate additional metrics associated with the comparative virtual model 624. For example, the comparator can, in addition to generative a visual comparison of the pre-op virtual model 620 and the post-op virtual model 622, generate a comparison of spinal loading of the spine in the pre-op virtual model 620 and the predicted post-op virtual model 622. The loading can be adjusted by the user to simulate different loading conditions. For dynamic loading, the comparator can perform dynamic simulations and provide comparisons between various models to evaluate how a surgical procedure will affect motion of the patient.

In some embodiments, the surgical plan review dashboard 600 is a dynamic interface displayed by a physician device for concurrently viewing multiple surgical plans and/or virtual models, such as the pre-op virtual model 620, the post-op virtual model 622, the comparative virtual model 624, and/or additional comparison models. The user can select the number of models and visual representations for concurrent display. In some embodiments, each model can be displayed in a separate window that enables the user to visually manipulate the model by, for example, panning, zooming, cropping, or otherwise manipulating the model. In some embodiments, the windows are synchronized so that rotation of a model in one window causes corresponding rotation of another model. This allows a user to view different models from the same perspective to visually compare differences between the models. In some embodiments, the surgical plan review dashboard 600 enables real-time viewing of model updates based on concurrently inputted position input. For example, if the user modifies a target post-operative metric as described below, one or more of the displayed models can be updated in real time to view those inputs immediately.

The examples of the pre-op virtual model 620, the post-op virtual model 622, and the comparative virtual model 624 shown in FIGS. 6A-6C illustrate the patient's lumbar spinal region and a simulated surgical intervention at the L4-L5 and L5-S1 levels. However, the pre-op virtual model 620, the post-po virtual model 622, and the comparative virtual model 624 can illustrate other regions of interest for a potential surgical intervention, depending on the particular surgical intervention included in the surgical plan. Moreover, the virtual models can include additional anatomical structures beyond those shown, such as additional bony structures, cartilage, soft tissue, vascular tissue, nervous tissue, etc.

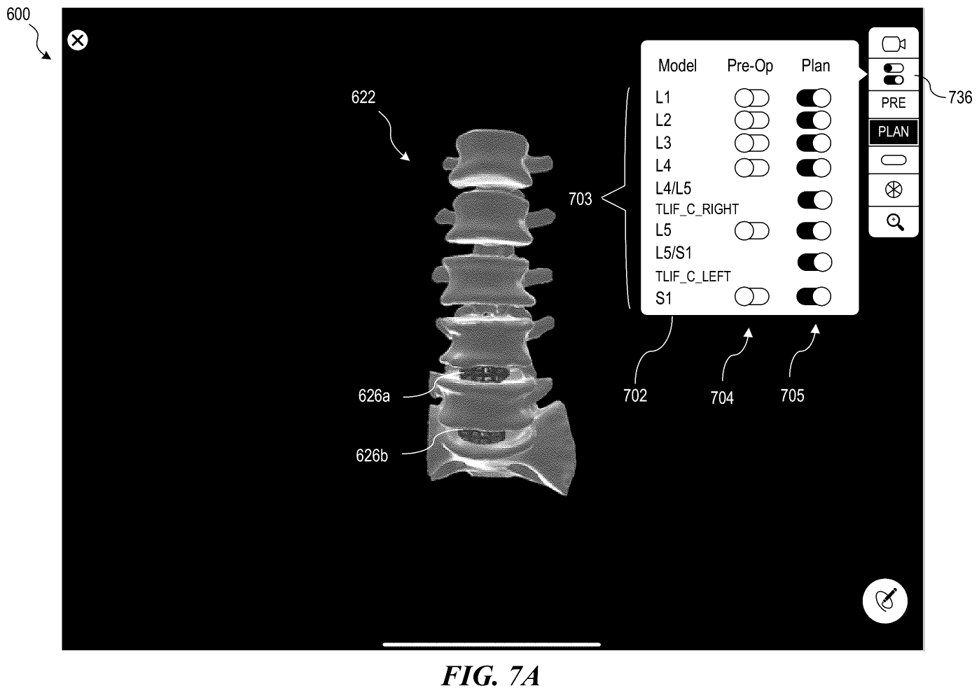

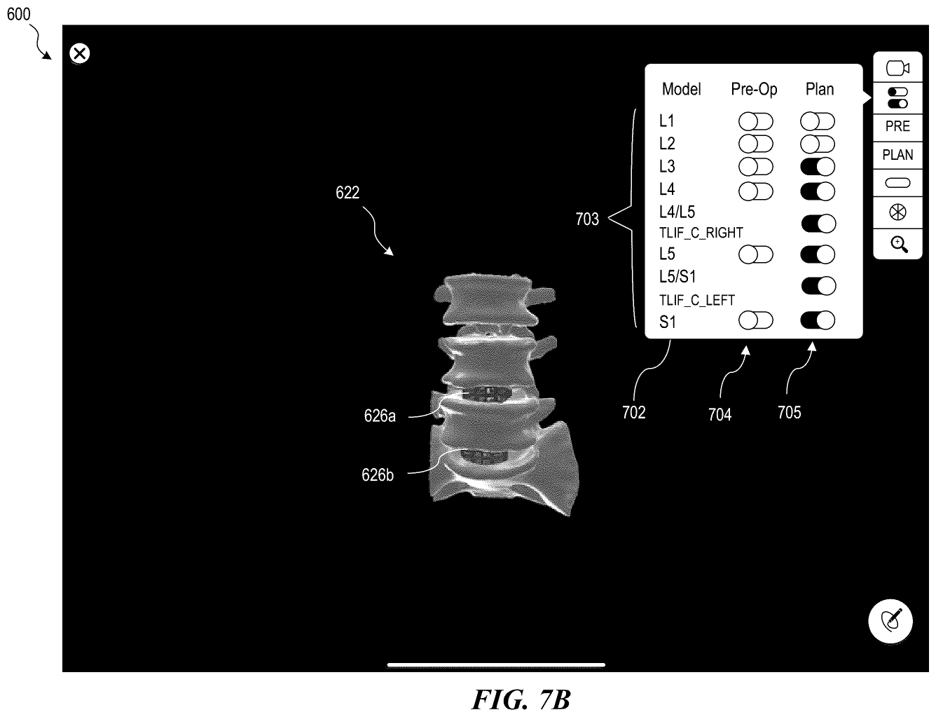

The surgical plan review dashboard 600 can also enable a user to selectively hide and show various portions/levels of the pre-op virtual model 620 (FIG. 6A) and the post-op virtual model 622 (FIG. 6B). For example, FIGS. 7A and 7B are third and fourth screenshots of the surgical plan review dashboard 600 and illustrate the ability of the program 125 to enable a user to selectively hide and show various portions/levels the virtual models. As shown in FIGS. 7A and 7B, the menu 630 includes a fourth selector 736 that, when selected, opens a selector window 702 for selectively hiding and showing various regions of the virtual models. In particular, the selector window 702 divides anatomy included in the pre-op virtual model 620 and/or the post-op virtual model 622 into a variety of discrete anatomical sub-regions 703 (shown in the illustrated embodiment as six vertebral levels and two virtual implants, although other sub-divisions are possible). The selector window 702 further includes a plurality of first selectors 704 that can be selectively toggled by a user to display or hide each discrete anatomical sub-region 703 of the pre-op virtual model 620, and a plurality of second selectors 705 that can be selectively toggled by a user to display or hide each discrete anatomical sub-region 703 of the post-op virtual model 622. The plurality of second selectors 705 further include options for selectively displaying or hiding the virtual implants 626 (shown as “L4/L5” and “L5/S1”). Accordingly, a user can selectively show or hide any combination of the discrete anatomical sub-regions 703, including both pre-operative and post-operative models of the discrete anatomical sub-regions 703, using the selector window 702.

Referring to FIG. 7A, each of the plurality of second selectors 705 is toggled “on,” and each of the plurality of first selectors 704 is toggled “off.” As a result, each of the discrete anatomical sub-regions 703 for the post-op virtual model 622 are displayed, and none of the discrete anatomical sub-regions 703 for the pre-op virtual model 620 are displayed. Turning to FIG. 7B, the second selectors 705 associated with the L1 and L2 sub-regions 703 have been toggled “off” as compared to the configuration shown in FIG. 7A, and so the L1 and L2 anatomical regions for the post-op virtual model 622 are no longer displayed. As one skilled in the art will appreciate from the disclosure herein, FIGS. 7A and 7B are provided by way of example to illustrate the ability of a user to selectively control which sub-regions 703 of the virtual models are displayed by the surgical plan review dashboard 600. Any combination of sub-regions 703 can be displayed using the selector window 702.

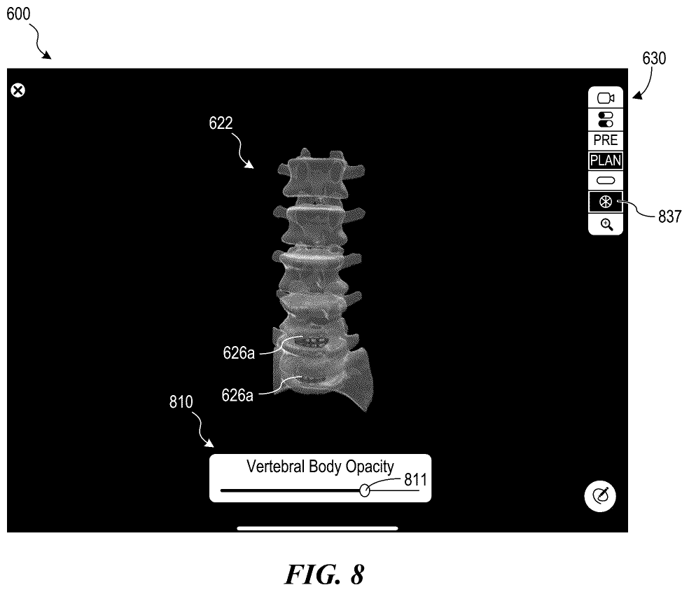

The surgical plan review dashboard 600 can also enable a user to selectively change an opacity or transparency of the displayed virtual model. For example, as shown in FIG. 8, which is a fifth screenshot of the surgical plan review dashboard 600, the menu 630 includes a fifth selector 837 that, when selected, opens a virtual model opacity window 810. The vertebral body opacity window 810 includes a slideable selector 811 that enables a user to adjust the opacity or transparency of the displayed virtual model. In the illustrated embodiment, the displayed virtual model is the post-op virtual model 622, although in other embodiments the displayed virtual model may be the pre-op virtual model 620 and/or the combined virtual model 624. In some embodiments, sliding the slideable selector 811 changes an opacity of the anatomy depicted in the displayed virtual model without changing an opacity of the virtual implants 626 shown in the displayed virtual model. In this way, the virtual model opacity window 810 facilitates better visualization of the virtual implants 626 by enabling a user to “see through” the patient anatomy.

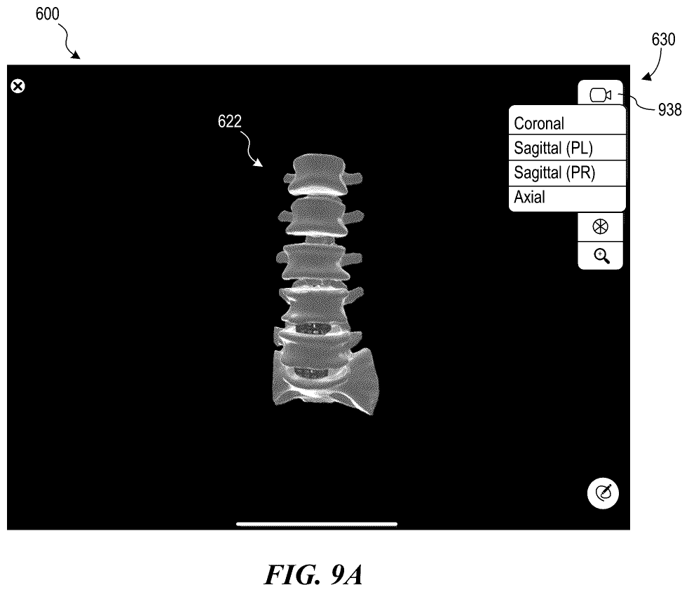

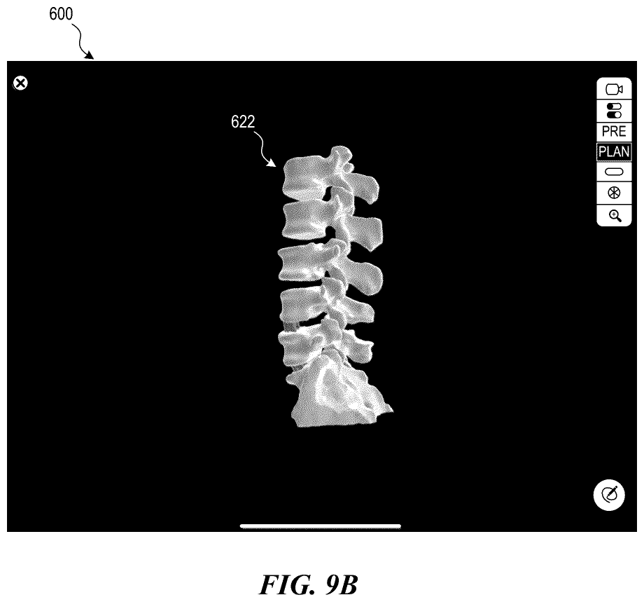

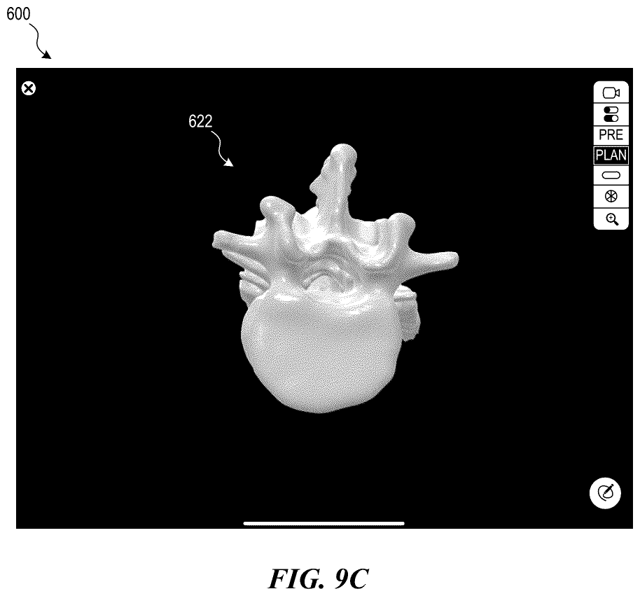

The surgical plan review dashboard 600 can also enable a user to view the virtual models from various orientations, as shown in FIGS. 9A-9C, which are sixth, seventh, and eight screenshots of the surgical plan review dashboard 600. For example, referring first to FIG. 9A, the menu 630 includes a sixth selector 938 that, when selected, enables a user to select between various views for the displayed virtual model. In the illustrated embodiment, the views include coronal, sagittal (PL) (e.g., sagittal left), sagittal (PR) (e.g., sagittal left), and axial/transverse. FIG. 9A illustrates a coronal view of the post-op virtual model 622, FIG. 9B illustrates a sagittal left-side view of the post-op virtual model 622, and FIG. 9C illustrates an axial view of the post-op virtual model 622. In some embodiments the program 125 may provide more or fewer views. Moreover, although FIGS. 9A-9C illustrate providing various views of the post-op virtual model 622, the program 125 can further provide the same views of the pre-op virtual model 620 and the combined virtual model 624. The program 125 can also provide views of just a portion of the virtual model, based on the selections made using the selector window 702 (FIGS. 7A and 7B).

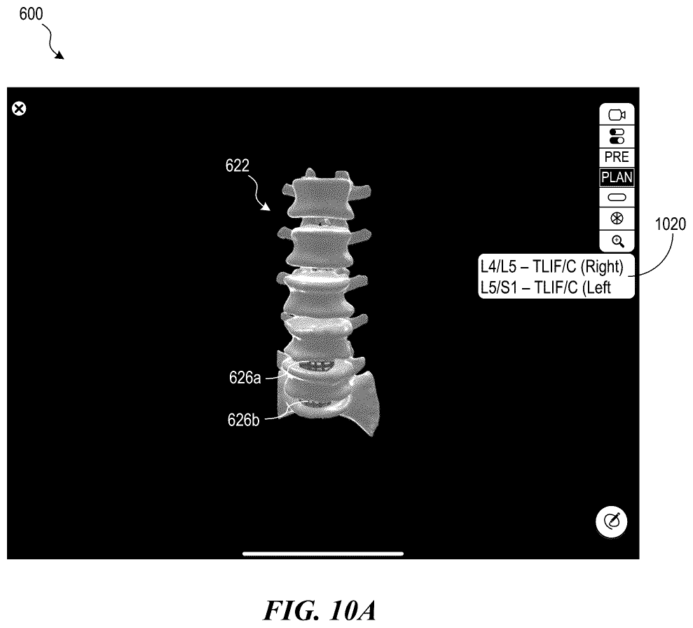

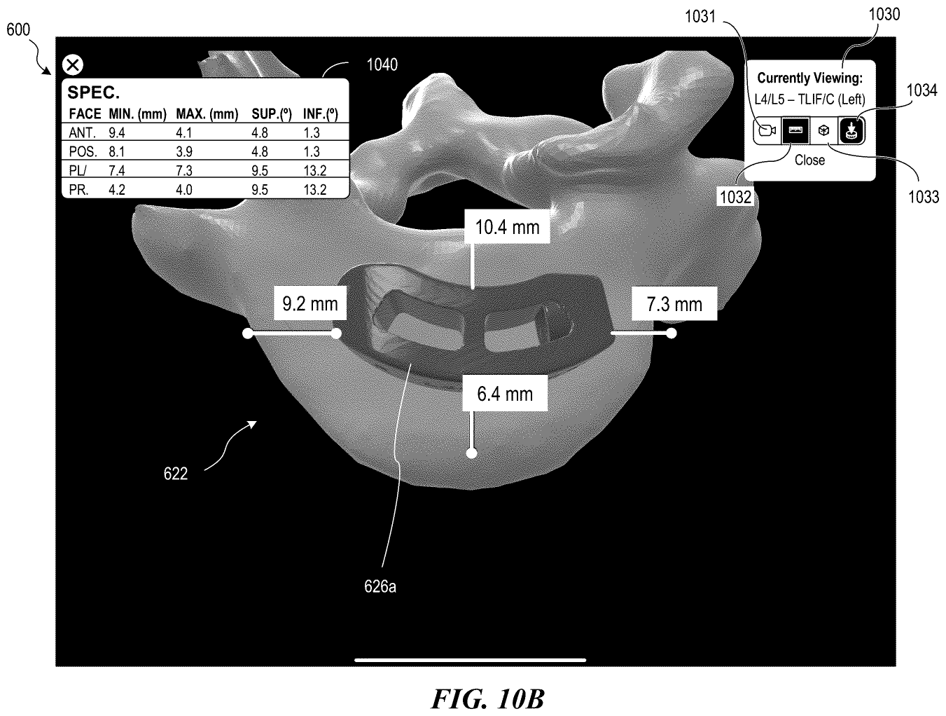

The surgical plan review dashboard can also enable a user to selectively display or hide the virtual implants 626, as shown in FIGS. 10A and 10B, which are ninth and tenth screenshots of the surgical plan review dashboard 600. In particular, a user can select to show or hide the first virtual implant 626a and the second virtual implant 626b using a virtual implant display selector 1020. In some embodiments, this feature is omitted because the same functionality is provided by the selector window 702, described with reference to FIGS. 7A and 7B. FIG. 10A illustrates a coronal view with both virtual implants 626 showing, and FIG. 10B illustrates an axial view with only the first virtual implant 626a visible.

In some embodiments, and as shown in FIG. 10B, a second menu 1030 is displayed to the user when the user is viewing the virtual implants 626 in the post op virtual model 622. A user can select various viewing options associated with the virtual implants 626 using the second menu 1030. For example, the user can use the second menu 1030 to select a camera view 1031 to display the virtual implant(s) 626 from various angles (e.g., sagittal left/right, coronal, and/or axial). The second menu 1030 can also include a measurement selector 1032 that can be selected/toggled on to display various measurements overlaid on the virtual model 622. For example, the measurements may include a distance of the implant 626 to the vertebral plate edges along the anterior, posterior, patient right and/or left sides (as shown in FIG. 10B), and/or other measurements. The second menu 1030 can also include a selector 1033 for changing the display of the superior vertebrae to give the user a better view of how the implant fits in the vertebral space (e.g., by changing an opacity of the superior vertebrae, by hiding the superior vertebrae, etc.). The second menu 1030 can also include an implant measurement selector 1034 that, when toggled on, causes an implant measurement table 1040 to be displayed. As shown, the implant measurement table 1040 can display various metrics of the virtual implant 626, such as, but not limited to, minimum and maximum heights and angles of each side of the virtual implant 626.

A user can also approve and/or provide suggested changes to the surgical plan using the surgical plan review dashboard 600. For example, FIG. 11 is an eleventh screenshot of the surgical plan review dashboard 600 and illustrates the ability of the program 125 to receive comments about the surgical plan from a user reviewing the surgical plan using the program 125. More specifically, FIG. 11 illustrates the surgical plan review dashboard 600 after the plan feedback selector 605 has been selected by a user. In response to the plan feedback selector 605 being selected, the surgical plan review dashboard 600 displays an “approve plan” option 1112 and a “suggest changes” option 1114. In the embodiment shown in FIG. 11, the suggest changes option 1114 has been selected by a user. As a result, and as shown in FIG. 11, the surgical plan review dashboard 600 opens a comments window 1115 and a user input mechanism 1116 (e.g., a touchscreen keyboard, a physical keyboard, a microphone, a stylus, etc.). A user (e.g., surgeon) can input feedback, suggestions, or comments about the surgical plan into the comments window 1115 using the user input mechanism 1116. The program 125 can then save the inputs (e.g., comments, target post-operative metrics, disapproved metrics, etc.) with the surgical plan, and optionally send the inputs to a remote computing system for surgical plan revision, as described above with respect to block 310 of the method 300 (FIG. 3). The embodiment shown in FIG. 12, which is a twelfth screenshot of the surgical plan review dashboard 600, illustrates the dashboard 600 following selection of the approve plan option 1112. In response to a user selecting the approve plan option 1112, the dashboard 600 opens an approval window 1215 that enables a user (e.g., surgeon) to certify and confirm that they approve the surgical plan. The program 125 can then send an indication that the user has approved the surgical plan to the remote computing system, as described with reference to block 308 of the method 300 (FIG. 3) and block 412 of the method 400 (FIG. 4).

As described in detail previously throughout this Detailed Description, a design platform (e.g., system 100 of FIG. 1) can design implants based on the approved surgical plan (e.g., if the implants were not designed and transmitted with the proposed surgical plan). For example, the design platform can design virtual implants that fit a virtual anatomical model of patient corresponding to the approved surgical plan such that the implants will produce the predicted postoperative anatomy. The design platform can generate CAD data, manufacturing data, manufacture instructions, or other data for a manufacturing system (e.g., manufacturing system 124 of FIG. 1).

As described above, FIGS. 5A-12 illustrate various aspects of the surgical plan review program 125 that enables a surgeon or other user to review, approve, and/or provide feedback on a proposed surgical plan. As described the surgical plan review program 125 can include a case display dashboard 500 (FIGS. 5A-5E) and a surgical plan review dashboard 600 (FIGS. 6A-12). As one skilled in the art will appreciate, the case display dashboard 500 and/or the surgical plan review dashboard 600 can include more or fewer functions than described above with respect to FIGS. 5A-12. Moreover, the case display dashboard 500 and/or the surgical plan review dashboard 600 can have alternative appearances than those shown in FIGS. 5A-12, including different configurations, color patterns, organizations, or the like.

Without intending to be bound by theory, the surgical plan review program 125, including the case display dashboard 500 and the surgical plan review dashboard 600, is expected to provide a number of advantages. For example, the case display dashboard 500 is expected to enable a healthcare clinic, surgeon, or other user to easily track and sort any number of unique patient cases. Moreover, the surgical plan review dashboard 600 is expected to enable a surgeon or other user to thoroughly review a proposed surgical plan for a patient, including predicted post-operative patient anatomy and metrics. The surgical plan review dashboard also enables the surgeon or other user to easily communicate any suggested feedback on or changes to the surgical plan, which can be transmitted back to the remote computing system as described above with reference to FIGS. 3 and 4. Accordingly, in various embodiments the surgical plan review program 125 described herein is expected to improve case management, surgeon review of proposed surgical plans, efficiency of surgeon review of proposed surgical plans, and/or patient outcomes.

As one skilled in the art will appreciate, any of the software modules described previously may be combined into a single software module for performing the operations described herein. Likewise, the software modules can be distributed across any combination of the computing systems and devices described herein, and are not limited to the express arrangements described herein. Accordingly, any of the operations described herein can be performed by any of the computing devices or systems described herein, unless expressly noted otherwise.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In some embodiments, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermediate components. Likewise, any two components so associated can also be viewed as being “operably connected,” or “operably coupled,” to each other to achieve the desired functionality, and any two components capable of being so associated can also be viewed as being “operably couplable” to each other to achieve the desired functionality. Specific examples of operably couplable include but are not limited to physically mateable and/or physically interacting components and/or wirelessly interactable and/or wirelessly interacting components and/or logically interacting and/or logically interactable components.

The embodiments, features, systems, devices, materials, methods and techniques described herein may, in some embodiments, be similar to any one or more of the embodiments, features, systems, devices, materials, methods and techniques described in the following:
U.S. application Ser. No. 16/048,167, filed on Jul. 27, 2018, titled “SYSTEMS AND METHODS FOR ASSISTING AND AUGMENTING SURGICAL PROCEDURES;”
U.S. application Ser. No. 16/242,877, filed on Jan. 8, 2019, titled “SYSTEMS AND METHODS OF ASSISTING A SURGEON WITH SCREW PLACEMENT DURING SPINAL SURGERY;”
U.S. application Ser. No. 16/207,116, filed on Dec. 1, 2018, titled “SYSTEMS AND METHODS FOR MULTI-PLANAR ORTHOPEDIC ALIGNMENT;”
U.S. application Ser. No. 16/352,699, filed on Mar. 13, 2019, titled “SYSTEMS AND METHODS FOR ORTHOPEDIC IMPLANT FIXATION;”
U.S. application Ser. No. 16/383,215, filed on Apr. 12, 2019, titled “SYSTEMS AND METHODS FOR ORTHOPEDIC IMPLANT FIXATION;”
U.S. application Ser. No. 16/569,494, filed on Sep. 12, 2019, titled “SYSTEMS AND METHODS FOR ORTHOPEDIC IMPLANTS;” and
U.S. application Ser. No. 16/699,447, filed Nov. 29, 2019, titled “SYSTEMS AND METHODS FOR ORTHOPEDIC IMPLANTS;”
U.S. application Ser. No. 16/735,222, filed Jan. 6, 2020, titled “PATIENT-SPECIFIC MEDICAL PROCEDURES AND DEVICES, AND ASSOCIATED SYSTEMS AND METHODS;”
U.S. application Ser. No. 16/987,113, filed Aug. 6, 2020, titled “PATIENT-SPECIFIC ARTIFICIAL DISCS, IMPLANTS AND ASSOCIATED SYSTEMS AND METHODS;”
U.S. application Ser. No. 16/990,810, filed Aug. 11, 2020, titled “LINKING PATIENT-SPECIFIC MEDICAL DEVICES WITH PATIENT-SPECIFIC DATA, AND ASSOCIATED SYSTEMS, DEVICES, AND METHODS;”
U.S. application Ser. No. 17/085,564, filed Oct. 30, 2020, titled “SYSTEMS AND METHODS FOR DESIGNING ORTHOPEDIC IMPLANTS BASED ON TISSUE CHARACTERISTICS;”
U.S. application Ser. No. 17/100,396, filed Nov. 20, 2020, titled “PATIENT-SPECIFIC VERTEBRAL IMPLANTS WITH POSITIONING FEATURES;”
U.S. application Ser. No. 17/342,439, filed Jun. 8, 2021, titled ““PATIENT-SPECIFIC MEDICAL PROCEDURES AND DEVICES, AND ASSOCIATED SYSTEMS AND METHODS;”
U.S. application Ser. No. 17/463,054, filed Aug. 31, 2021, titled “BLOCKCHAIN MANAGED MEDICAL IMPLANTS;”
U.S. application Ser. No. 17/518,524, filed Nov. 3, 2021, titled “PATIENT-SPECIFIC ARTHROPLASTY DEVICES AND ASSOCIATED SYSTEMS AND METHODS;”
U.S. application Ser. No. 17/531,417, filed Nov. 19, 2021, titled “PATIENT-SPECIFIC JIG FOR PERSONALIZED SURGERY;”
U.S. application Ser. No. 17/678,874, filed Feb. 23, 2022, titled “NON-FUNGIBLE TOKEN SYSTEMS AND METHODS FOR STORING AND ACCESSING HEALTHCARE DATA;”
U.S. application Ser. No. 17/835,777, filed Jun. 8, 2022, titled “PATIENT-SPECIFIC EXPANDABLE INTERVERTEBRAL IMPLANTS;”
U.S. application Ser. No. 17/842,242, filed Jun. 16, 2022, titled “PATIENT-SPECIFIC ANTERIOR PLATE IMPLANTS;”
U.S. application Ser. No. 17/851,487, filed Jun. 28, 2022, titled “PATIENT-SPECIFIC ADJUSTMENT OF SPINAL IMPLANTS, AND ASSOCIATED SYSTEMS AND METHODS;”
U.S. application Ser. No. 17/856,625, filed Jul. 1, 2022, titled “SPINAL IMPLANTS FOR MESH NETWORKS;”
U.S. application Ser. No. 17/867,621, filed Jul. 18, 2022, titled “PATIENT-SPECIFIC SACROILIAC IMPLANT, AND ASSOCIATED SYSTEMS AND METHODS;”
U.S. application Ser. No. 17/868,729, filed Jul. 19, 2022, titled “SYSTEMS FOR PREDICTING INTRAOPERATIVE PATIENT MOBILITY AND IDENTIFYING MOBILITY-RELATED SURGICAL STEPS;”
U.S. application Ser. No. 17/978,673, filed Nov. 1, 2022, titled “SPINAL IMPLANTS AND SURGICAL PROCEDURES WITH REDUCED SUBSIDENCE, AND ASSOCIATED SYSTEMS AND METHODS;”
U.S. application Ser. No. 17/978,746, filed Nov. 1, 2022, titled “PATIENT-SPECIFIC SPINAL INSTRUMENTS FOR IMPLANTING IMPLANTS AND DECOMPRESSION PROCEDURES;”
U.S. application Ser. No. 18/102,444, filed Jan. 27, 2023, titled “TECHNIQUES TO MAP THREE-DIMENSIONAL HUMAN ANATOMY DATA TO TWO-DIMENSIONAL HUMAN ANATOMY DATA;”
U.S. application Ser. No. 18/113,573, filed Feb. 24, 2023, titled “PATIENT-SPECIFIC IMPLANT DESIGN AND MANUFACTURING SYSTEM WITH A DIGITAL FILING CABINET;”
U.S. application Ser. No. 18/120,979, filed Mar. 13, 2023, titled “MULTI-STAGE PATIENT-SPECIFIC SURGICAL PLANS AND SYSTEMS AND METHODS FOR CREATING AND IMPLEMENTING THE SAME;”
U.S. application Ser. No. 18/455,881, filed Aug. 25, 2023, titled “SYSTEMS AND METHODS FOR GENERATING MULTIPLE PATIENT-SPECIFIC SURGICAL PLANS AND MANUFACTURING PATIENT-SPECIFIC IMPLANTS;”
U.S. application Ser. No. 17/951,085, filed Sep. 22, 2022, titled “SYSTEMS FOR MANUFACTURING AND PRE-OPERATIVE INSPECTING OF PATIENT-SPECIFIC IMPLANTS;”
U.S. Application No. 63/420,279, filed Oct. 28, 2022, titled “SYSTEMS AND METHODS FOR SELECTING, REVIEWING, MODIFYING, AND/OR APPROVING SURGICAL PLANS;”
U.S. Application No. 63/387,009, filed Dec. 12, 2022, titled “PATIENT-SPECIFIC IMPLANT DESIGN AND MANUFACTURING SYSTEM WITH A REGULATORY AND REIMBURSEMENT MANAGER;”
U.S. Application No. 63/436,860, filed Jan. 3, 2023, titled “PATIENT-SPECIFIC SPINAL FUSION DEVICES AND ASSOCIATED SYSTEMS AND METHODS;”
U.S. Application No. 63/437,966, filed Jan. 9, 2023, titled “SYSTEM FOR EDGE CASE PATHOLOGY IDENTIFICATION AND IMPLANT MANUFACTURING;”
U.S. Application No. 63/437,975, filed Jan. 9, 2023, titled “SYSTEM FOR MODELING PATIENT SPINAL CHANGES;”
U.S. Application No. 63/522,815, filed Jun. 23, 2023, titled “SYSTEMS AND METHODS FOR DIAGNOSING SPINAL CONDITIONS AND DETERMINING TREATMENT OF THE SAME;”
U.S. Application No. 63/530,427, filed Aug. 2, 2023, titled “MEDICAL DEVICE INSERTER INSTRUMENTS WITH RETRACTABLE COUPLING ELEMENTS AND METHODS OF USING THE SAME;” and
U.S. Application No. 63/542,264, filed Oct. 3, 2023, titled “PATIENT-SPECIFIC SURGICAL POSITIONING GUIDES AND METHODS OF MAKING AND USING THE SAME.”

All of the above-identified patents and applications are incorporated by reference in their entireties. In addition, the embodiments, features, systems, devices, materials, methods and techniques described herein may, in certain embodiments, be applied to or used in connection with any one or more of the embodiments, features, systems, devices, or other matter.

The ranges disclosed herein also encompass any and all overlap, sub-ranges, and combinations thereof. Language such as “up to,” “at least,” “greater than,” “less than,” “between,” or the like includes the number recited. Numbers preceded by a term such as “approximately,” “about,” and “substantially” as used herein include the recited numbers (e.g., about 10%=10%), and also represent an amount close to the stated amount that still performs a desired function or achieves a desired result. For example, the terms “approximately,” “about,” and “substantially” may refer to an amount that is within less than 10% of, within less than 5% of, within less than 1% of, within less than 0.1% of, and within less than 0.01% of the stated amount.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting.

I/We claim:

1. A method for designing one or more patient-specific spinal implants, the method comprising:
building a first spine model representing pre-operative spinal anatomy of a patient, the first spine model including vertebral elements;
generating a second spine model representing predicted post-operative spinal anatomy of the patient based on one or more surgical corrections;
generating at least one visual comparison of the first spine model and the second spine model using a comparator of a design platform;
linking the first spine model and the second spine model to a dynamic interface displayed by a physician device for concurrently viewing the first spine model, the second spine model, and the at least one visual comparison, wherein the dynamic-viewer interface is configured to
display the first spine model, the second spine model, and the at least one visual comparison,
dynamically display one or more spinal metrics corresponding to anatomical elements selected for viewing by user, and
receive physician input that is inputted into the dynamic interface;
in response to the received physician input including one or more target post-operative metrics, generating, via the design platform, a modified second spine model representing predicted post-operative anatomy of the patient for the target post-operative metrics, wherein the modified second spine model and at least one visual comparison of the first spine model and the modified second spine model generated by the comparator is viewable using the dynamic interface; and
designing, using the design platform, one or more spinal implants based on one of the second spine model or the modified second spine model approved by the user such that the one or more spinal implants produce the predicted post-operative anatomy of the approved second spine model or the modified second spine model when implanted in the patient.

2. The method of claim 1, further comprising digitally overlaying, using the comparator, the first and second models to generate the at least one visual comparison.

3. The method of claim 1, wherein linking of the first spine model and the second spine model to the dynamic interface enables real-time viewing of model updates based on concurrently inputted physician input.

4. The method of claim 1, wherein the linking is across a wide area network between the design platform and the user device.

5. The method of claim 1, further comprising performing one or more clinical checks on the approved second spine model or the approved modified second spine model.

6. The method of claim 1, wherein the linking enables panning, rotating, and/or zooming of one or more of the first or second spine models for visual comparison via the dynamic interface.

7. The method of claim 1, further comprising synchronizing panning, rotating, and/or zooming of the first spine model and the second spine model for display by the dynamic interface.

8. The method of claim 1, wherein the at least one visual comparison displayed in the dynamic interface illustrates spinal loading differences between the spine of the patient in the first spine model and the spine of the patient in the second spine model.

9. A system for designing patient-specific surgical plans, the system comprising:
a first virtual model of pre-operative anatomy of a patient;
a second virtual model of predicted post-operative anatomy of the patient;
a third virtual model of the predicted post-operative anatomy overlaid over the pre-operative anatomy;
a processor; and
a memory storing non-transitory instructions for a surgical plan review program, wherein, the surgical plan review program enables a user to view the first virtual model, the second virtual model, and the third virtual model.

10. The system of claim 9 wherein the surgical plan review program includes one or more virtual model selectors that enable a user to selectively toggle between viewing the first virtual model, the second virtual model, and the third virtual model.

11. The system of claim 9 wherein the surgical plan review program includes one or more orientation selectors that enable a user to selectively change a viewing orientation of the first virtual model, the second virtual model, and the third virtual model.

12. The system of claim 9 wherein the third virtual model shows the predicted post-operative anatomy in a different color and/or pattern than the pre-operative anatomy.

13. The system of claim 9 wherein the surgical plan review program further displays anatomical metrics associated with whichever of the first virtual model, the second virtual model, or the third virtual model is being displayed.

14. The system of claim 9 wherein each virtual model includes a plurality of discrete sub-regions, and wherein the surgical plan review program provides one or more selectors for selectively showing and hiding each of the individual sub-regions.

15. The system of claim 14 wherein the discrete sub-regions are individual vertebral levels.

16. A non-transitory computer readable medium storing instructions associated with a surgical plan review program for reviewing patient-specific surgical plans using a computing device, wherein the instructions include first instructions and second instructions, and wherein:
when the first instructions are executed, the computing device displays, via a display screen, a first digital dashboard, wherein the first digital dashboard includes a plurality of patient cases associated with a plurality of patients, wherein each of the plurality of patient cases includes a proposed surgical plan for treating the corresponding patient; and
when the second instructions are executed, the computing device displays, via the display screen, a second digital dashboard, wherein the second digital dashboard includes:
a menu for selecting for viewing one or more features associated with the proposed surgical plan for a particular patient of the plurality of patients, wherein the one or more features include a virtual model of predicted post operative anatomy of the patient if the surgical plan were to be performed and predicted post operative anatomical metrics associated with the predicted post operative anatomy, and
a plan feedback selector, wherein, in response to a user selecting the plan feedback selector, the second digital dashboard permits the user to provide feedback on the proposed surgical plan and/or approve the proposed surgical plan.

17. The non-transitory computer readable medium of claim 16 wherein the second instructions are executed in response to a user selecting for viewing the proposed surgical plan for the particular patient in the first dashboard.

18. The non-transitory computer readable medium of claim 16 wherein the second digital dashboard is interactive and configured to receive user input via an input mechanism, and wherein, in response to the user input the second digital dashboard changes the one or more features associated with the surgical plan displayed for user review.

19. The non-transitory computer readable medium of claim 16 wherein the one or more features associated with the surgical plan further include one or more virtual implants.

20. The non-transitory computer readable medium of claim 16 wherein the virtual model includes one or more virtual implants.

21. The non-transitory computer readable medium of claim 16 wherein the virtual model is a first virtual model, and wherein, the one or more features associated with the surgical plan further include a second virtual model of pre-operative patient anatomy.

22. The non-transitory computer readable medium of claim 21 wherein the one or more features associated with the surgical plan further include a third virtual model, wherein the third virtual model includes both the first virtual model and the second virtual model to compare predicted post operative patient anatomy with pre-operative patient anatomy.

23. The non-transitory computer readable medium of claim 16 wherein the predicted post operative anatomical metrics include coronal parameters, sagittal parameters, pelvic parameters, Cobb angles, shoulder tilt, iliolumbar angles, coronal balance, lordosis angles, and/or intervertebral space height.

24. The non-transitory computer readable medium of claim 16 wherein the virtual model includes a plurality of discrete sub-regions, and wherein the menu includes a selector for selectively showing and hiding each individual sub-region of the plurality of discrete sub-regions.

25. The non-transitory computer readable medium of claim 24 wherein individual sub-regions of the plurality of discrete sub-regions correspond to individual vertebral levels.

26. The non-transitory computer readable medium of claim 16 wherein the menu includes a selector for changing a transparency of the virtual model.

27. The non-transitory computer readable medium of claim 16 wherein the menu includes a selector for changing a viewing orientation of the virtual model.

28. The non-transitory computer readable medium of claim 27 wherein the selector provides a plurality of viewing orientations, the plurality of viewing orientations including a coronal view, a sagittal view, and an axial view.

29. The non-transitory computer readable of claim 16 wherein the surgical review program is configured to associate any feedback received from the user via the plan feedback selector with the proposed surgical plan.

30. A computer-implemented method for reviewing a patient-specific surgical plan for a patient using a surgical plan review program, the method comprising:
displaying a first digital dashboard, wherein the first digital dashboard includes a plurality of patient cases associated with a plurality of patients, wherein each of the plurality of patient cases includes a proposed surgical plan for treating the corresponding patient;
receiving, from a user, a selection of a particular patient case from the plurality of patient cases;
in response to receiving the selection of a particular patient case, displaying a second digital dashboard, wherein the second digital dashboard includes:
a menu for selecting for viewing one or more features associated with the proposed surgical plan for the particular patient, wherein the one or more features include a virtual model of predicted post operative anatomy of the patient if the surgical plan were to be performed and predicted post operative anatomical metrics associated with the predicted post operative anatomy, and
a plan feedback selector for receiving feedback on the proposed surgical plan and/or approving the proposed surgical plan;
receiving a selection of the plan feedback selector;
in response to receiving a selection of the plan feedback selector, displaying a field for receiving comments from the user and/or displaying a selector for approving the proposed surgical plan; and
receiving, from the user, feedback on the proposed surgical plan via the field and/or approval of the proposed surgical plan via the selector.

31. The computer-implemented method of claim 30 wherein, before receiving a selection of the plan feedback selector, the method comprises:
receiving, via the menu, a selection of the one or more features associated with the surgical plan; and
in response to receiving the selection, updating the second digital dashboard to display for viewing the selected feature.

32. The computer-implemented method of claim 30 wherein the one or more features associated with the surgical plan further include one or more virtual implants.

33. The computer-implemented method of claim 30 wherein the virtual model is a first virtual model, and wherein, the one or more features associated with the surgical plan further include a second virtual model of pre-operative patient anatomy.

34. The computer-implemented method of claim 33 wherein the one or more features associated with the surgical plan further include a third virtual model, wherein the third virtual model includes both the first virtual model and the second virtual model to compare predicted post operative patient anatomy with pre-operative patient anatomy.

35. The computer-implemented method of claim 30 wherein the predicted post operative anatomical metrics include coronal parameters, sagittal parameters, pelvic parameters, Cobb angles, shoulder tilt, iliolumbar angles, coronal balance, lordosis angles, and/or intervertebral space height.

36. The computer-implemented method of claim 30 wherein the virtual model includes a plurality of discrete sub-regions, and wherein the menu includes a selector for selectively showing and hiding each individual sub-region of the plurality of discrete sub-regions.
